SEQUENCE LISTING (83 sequences)

SEQ ID NO: 1 - DNA, 975 bp, Homo sapiens
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 2 - DNA, 975 bp, Homo sapiens
  MISC_FEATURE (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 3 - DNA, 975 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 4 - DNA, 975 bp, Homo sapiens
  MISC_FEATURE (194)..(194): t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccgnngtggtggctgatcggcgcagagtccgtgtgagactgctaagcggcgacacgtatgaggccgtggtcacagctgtggatcccgtggcagnnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 5 - DNA, 975 bp, Homo sapiens
  misc_feature (193)..(195): n = a, t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnnngtggtggctgatcggcgcagagtccgtgtgagactgctaagcggcgacacgtatgaggccgtggtcacagctgtggatcccgtggcannnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacnnnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 6 - DNA, 675 bp, Homo sapiens
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 7 - DNA, 675 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 8 - DNA, 675 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 9 - DNA, 675 bp, Homo sapiens
  misc_feature (194)..(194): n = t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccgnngtggtggctgatcggcgcagagtccgtgtgagactgctaagcggcgacacgtatgaggccgtggtcacagctgtggatcccgtggcagnnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 10 - DNA, 675 bp, Homo sapiens
  misc_feature (193)..(195): n = a, t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnnngtggtggctgatcggcgcagagtccgtgtgagactgctaagcggcgacacgtatgaggccgtggtcacagctgtggatcccgtggcannnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacnnnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 11 - DNA, 963 bp, Homo sapiens
ccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 12 - DNA, 975 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 13 - DNA, 975 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccagcggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 14 - DNA, 663 bp, Homo sapiens
ccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 15 - DNA, 675 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 16 - DNA, 675 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcatcgtggggaaaagaagaattcctcctccggaatcagtgggtcccag

SEQ ID NO: 17 - DNA, 636 bp, Homo sapiens
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggg

SEQ ID NO: 18 - DNA, 636 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 19 - DNA, 636 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcatcgtggg

SEQ ID NO: 20 - DNA, 636 bp, Homo sapiens
  misc_feature (194)..(194): n = t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccgnngtggtggctgatcggcgcagagtccgtgtgagactgctaagcggcgacacgtatgaggccgtggtcacagctgtggatcccgtggcagnnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 21 - DNA, 636 bp, Homo sapiens
  misc_feature (193)..(195): n = a, t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnnngtggtggctgatcggcgcagagtccgtgtgagactgctaagcggcgacacgtatgaggccgtggtcacagctgtggatcccgtggcannnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacnnnggaggtcccctggttaacctggatggggaggtgattggagtgaacaccatgaaggtcacagctggaatctcctttgccatcccttctgatcgtcttcgagagtttctgcatcgtggg

SEQ ID NO: 22 - DNA, 624 bp, Homo sapiens
ccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcatcgtggg

SEQ ID NO: 23 - DNA, 636 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 24 - DNA, 636 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcatcgtggg

SEQ ID NO: 25 - DNA, 630 bp, Homo sapiens
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcat

SEQ ID NO: 26 - DNA, 630 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcat

SEQ ID NO: 27 - DNA, 630 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcat

SEQ ID NO: 28 - DNA, 630 bp, Homo sapiens
  misc_feature (194)..(194): n = t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccgnngtggtggctgatcggcgcagagtccgtgtgagactgctaagcggcgacacgtatgaggccgtggtcacagctgtggatcccgtggcagnnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcat

SEQ ID NO: 29 - DNA, 630 bp, Homo sapiens
  misc_feature (193)..(195): n = a, t, g, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnnngtggtggctgatcggcgcagagtccgtgtgagactgctaagcggcgacacgtatgaggccgtggtcacagctgtggatcccgtggcannnatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaacnnnggaggtcccctggttaacctggatggggaggtgattggagtgaacaccatgaaggtcacagctggaatctcctttgccatcccttctgatcgtcttcgagagtttctgcat

SEQ ID NO: 30 - DNA, 636 bp, Homo sapiens
  misc_feature (160)..(162): n = Deleted Nucleic Acids
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtgnnngctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 31 - DNA, 636 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagannnctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 32 - DNA, 636 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttnnngttgccatgggaagtccctttgcactgcagaacacgatcacatccggcattgttagctctgctcagcgtccagccagagacctgggactcccccaaaccaatgtggaatacattcaaactgatgcagctattgattttggaaacagnggaggtcccctggttaacctggatggggaggtgattggagtgaacaccatgaaggtcacagctggaatctcctttgccatcccttctgatcgtcttcgagagtttctgcatcgtggg

SEQ ID NO: 33 - DNA, 636 bp, Homo sapiens
  misc_feature (193)..(193): n = t, c
gccgtccctagcccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtccccnnnttaacctggatggggaggtgattggagtgaacaccatgaaggtcacagctggaatctcctttgccatcccttctgatcgtcttcgagagtttctgcatcgtggg

SEQ ID NO: 34 - DNA, 618 bp, Homo sapiens
ccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgcccatgtggtggctgatcggcgcagagtccgtgtgagactgctaagcggcgacacgtatgaggccgtggtcacagctgtggatcccgtggcagacatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactctggaggtcccctggttaacctggatggggaggtgattggagtgaacaccatgaaggtcacagctggaatctcctttgccatcccttctgatcgtcttcgagagtttctgcat

SEQ ID NO: 35 - DNA, 630 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcat

SEQ ID NO: 36 - DNA, 636 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtgnnngctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 37 - DNA, 636 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagannnctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 38 - DNA, 636 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttnnngttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtcccctggttaacctggatggggaggtgattggagtgaacaccatgaaggtcacagctggaatctcctttgccatcccttctgatcgtcttcgagagtttctgcatcgtggg

SEQ ID NO: 39 - DNA, 636 bp, Homo sapiens
  misc_feature (1)..(12): n = Cleaved Nucleic Acids
nnnnnnnnnnnnccgccgcccgcttctccccggagtcagtacaacttcatcgcagatgtggtggagaagacagcacctgccgtggtctatatcgagatcctggaccggcaccctttcttgggccgcgaggtccctatctcgaacggctcaggattcgtggtggctgccgatgggctcattgtcaccaacgccnangtggtggctgatcggcgcagagtccgtgtgagactgctaagcggcgacacgtatgaggccgtggtcacagctgtggatcccgtggcaganatcgcaacgctgaggattcagactaaggagcctctccccacgctgcctctgggacgctcagctgatgtccggcaaggggagtttgttgttgccatgggaagtccctttgcactgcagaacacgatcacatccggcattgttagctctgctcagcgtccagccagagacctgggactcccccaaaccaatgtggaatacattcaaactgatgcagctattgattttggaaactcnggaggtccccnnnttaacctggatggggaggtgattggagtgaacaccatgaaggtcacagctggaatctcctttgccatcccttctgatcgtcttcgagagtttctgcatcgtggg

SEQ ID NO: 40 - DNA, 300 bp, Homo sapiens
cggcgctacattggggtgatgatgctgaccctgagtcccagcatccttgctgaactacagcttcgagaaccaagctttcccgatgttcagcatggtgtactcatccataaagtcatcctgggctcccctgcacaccgggctggtctgcggcctggtgatgtgattttggccattggggagcagatggtacaaaatgctgaagatgtttatgaagctgttcgaacccaatcccagttggcagtgcagatccggcggggacgagaaacactgaccttatatgtgacccctgaggtcacagaa

SEQ ID NO: 41 - DNA, 12 bp, Homo sapiens
gccgtccctagc

SEQ ID NO: 42 - DNA, 2589 bp, Homo sapiens
tctaagtagtatcttggaaattcagagagatactcatcctacctgaatataaactgagataaatccagtaaagaaagtgtagtaaattctacataagagtctatcattgatttcttttggtggtaaaaatcttagttcatgtgaagaaatttcatgtgaatgttttagctatcaaacagcactgtcacctactcatgcacaaaactgcctcccaaagacttttcccaggtccctcgtatcaaaacattaagagtataatggaagatagcacgatcttgtcagattggacaaacagcaacaaacaaaaaatgaagtatgacttttcctgtgaactctacagaatgtctacatattcaactttccccgccggggtgcctgtctcagaaaggagtcttgctcgtgctggtttttattatactggtgtgaatgacaaggtcaaatgcttctgttgtggcctgatgctggataactggaaactaggagacagtcctattcaaaagcataaacagctatatcctagctgtagctttattcagaatctggtttcagctagtctgggatccacctctaagaatacgtctccaatgagaaacagttttgcacattcattatctcccaccttggaacatagtagcttgttcagtggttcttactccagcctttctccaaaccctcttaattctagagcagttgaagacatctcttcatcgaggactaacccctacagttatgcaatgagtactgaagaagccagatttcttacctaccatatgtggccattaacttttttgtcaccatcagaattggcaagagctggtttttattatataggacctggagatagggtagcctgctttgcctgtggtgggaagctcagtaactgggaaccaaaggatgatgctatgtcagaacaccggaggcattttcccaactgtccatttttggaaaattctctagaaactctgaggtttagcatttcaaatctgagcatgcagacacatgcagctcgaatgagaacatttatgtactggccatctagtgttccagttcagcctgagcagcttgcaagtgctggtttttattatgtgggtcgcaatgatgatgtcaaatgcttttgttgtgatggtggcttgaggtgttgggaatctggagatgatccatgggtagaacatgccaagtggtttccaaggtgtgagttcttgatacgaatgaaaggccaagagtttgttgatgagattcaaggtagatatcctcatcttcttgaacagctgttgtcaacttcagataccactggagaagaaaatgctgacccaccaattattcattttggacctggagaaagttcttcagaagatgctgtcatgatgaatacacctgtggttaaatctgccttggaaatgggctttaatagagacctggtgaaacaaacagttcaaagtaaaatcctgacaactggagagaactataaaacagttaatgatattgtgtcagcacttctaaatgctgaagatgaaaaaagagaggaggagaaggaaaaacaagctgaagaaatggcatcagatgatttgtcattaattcggaagaacagaatggctctctttcaacaattgacatgtgtgcttcctatcctggataatcttttaaaggccaatgtaattaataaacaggaacatgatattattaaacaaaaaacacagatacctttacaagcgagagaactgattgataccattttggttaaaggaaatgctgcggccaacatcttcaaaaactgtctaaaagaaattgactctacattgtataagaacttatttgtggataagaatatgaagtatattccaacagaagatgtttcaggtctgtcactggaagaacaattgaggaggttgcaagaagaacgaacttgtaaagtgtgtatggacaaagaagtttctgttgtatttattccttgtggtcatctggtagtatgccaggaatgtgccccttctctaagaaaatgccctatttgcaggggtataatcaagggtactgttcgtacatttctctcttaaagaaaaatagtctatattttaacctgcataaaaaggtctttaaaatattgttgaacacttgaagccatctaaagtaaaaagggaattatgagtttttcaattagtaacattcatgttctagtctgctttggtactaataatcttgtttctgaaaagatggtatcatatatttaatcttaatctgtttatttacaagggaagatttatgtttggtgaactatattagtatgtatgtgtacctaagggagtagtgtcactgcttgttatgcatcatttcaggagttactggatttgttgttctttcagaaagctttgaatactaaattatagtgtagaaaagaactggaaaccaggaactctggagttcatcagagttatggtgccgaattgtctttggtgcttttcacttgtgttttaaaataaggatttttctcttatttctccccctagtttgtgagaaacatctcaataaagtgctttaaaaaaaaaaaaaaaaaaaaaaaaaaaaaaaaaaaaaaaaaaaaaaaaaaaaaaaaaa

SEQ ID NO: 43 - DNA, 2589 bp, Homo sapiens; identical to SEQ ID NO: 42

SEQ ID NO: 44 - protein, 325 aa, Homo sapiens
AVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 45 - protein, 325 aa, Homo sapiens
  MISC_FEATURE (65)..(65): Xaa = His, Lys, Arg, Phe, Tyr, Trp
AVPSPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 46 - protein, 325 aa, Homo sapiens; identical to SEQ ID NO: 45
  MISC_FEATURE (65)..(65): Xaa = Ala, Asp, Asn, Cys, Glu, Gln, Gly, Ile, Leu, Met, Pro, Ser, Thr, Val

SEQ ID NO: 47 - protein, 325 aa, Homo sapiens; identical to SEQ ID NO: 45
  MISC_FEATURE (65)..(65): Xaa = Ala, Cys, Asp, Glu, Phe, Gly, His, Ile, Lys, Leu, Met, Asn, Pro, Gln, Arg, Ser, Thr, Val, Trp, Tyr

SEQ ID NO: 48 - protein, 225 aa, Homo sapiens
AVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRGEKKNSSSGISGSQ

SEQ ID NO: 49 - protein, 225 aa, Homo sapiens
  MISC_FEATURE (65)..(65): Xaa = His, Lys, Arg, Phe, Tyr, Trp
AVPSPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRGEKKNSSSGISGSQ

SEQ ID NO: 50 - protein, 225 aa, Homo sapiens; identical to SEQ ID NO: 49
  MISC_FEATURE (65)..(65): Xaa = Ala, Asp, Asn, Cys, Glu, Gly, Ile, Leu, Met, Pro, Ser, Thr, Val

SEQ ID NO: 51 - protein, 225 aa, Homo sapiens; identical to SEQ ID NO: 49
  MISC_FEATURE (65)..(65): Xaa = Ala, Cys, Asp, Glu, Phe, Gly, His, Ile, Lys, Leu, Met, Asn, Pro, Gln, Arg, Ser, Thr, Val, Trp, Tyr

SEQ ID NO: 52 - protein, 321 aa, Homo sapiens
PPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 53 - protein, 325 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRGEKKNSSSGISGSQRRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 54 - protein, 221 aa, Homo sapiens
PPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRGEKKNSSSGISGSQ

SEQ ID NO: 55 - protein, 225 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRGEKKNSSSGISGSQ

SEQ ID NO: 56 - protein, 212 aa, Homo sapiens
AVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRG

SEQ ID NO: 57 - protein, 212 aa, Homo sapiens
  MISC_FEATURE (65)..(65): Xaa = His, Lys, Arg, Phe, Tyr, Trp
AVPSPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 58 - protein, 212 aa, Homo sapiens; identical to SEQ ID NO: 57
  MISC_FEATURE (65)..(65): Xaa = Ala, Asp, Asn, Cys, Glu, Gln, Gly, Ile, Leu, Met, Pro, Ser, Thr, Val

SEQ ID NO: 59 - protein, 212 aa, Homo sapiens; identical to SEQ ID NO: 57
  MISC_FEATURE (65)..(65): Xaa = Ala, Cys, Asp, Glu, Phe, Gly, His, Ile, Lys, Leu, Met, Asn, Pro, Gln, Arg, Ser, Thr, Val, Trp, Tyr

SEQ ID NO: 60 - protein, 208 aa, Homo sapiens
PPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLHRG

SEQ ID NO: 61 - protein, 212 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 62 - protein, 210 aa, Homo sapiens
AVPSPPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLH

SEQ ID NO: 63 - protein, 210 aa, Homo sapiens
  MISC_FEATURE (65)..(65): Xaa = His, Lys, Arg, Phe, Tyr, Trp
AVPSPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLH

SEQ ID NO: 64 - protein, 210 aa, Homo sapiens; identical to SEQ ID NO: 63
  MISC_FEATURE (65)..(65): Xaa = Ala, Asp, Asn, Cys, Glu, Gln, Gly, Ile, Leu, Met, Pro, Ser, Thr, Val

SEQ ID NO: 65 - protein, 210 aa, Homo sapiens; identical to SEQ ID NO: 63
  MISC_FEATURE (65)..(65): Xaa = Ala, Cys, Asp, Glu, Phe, Gly, His, Ile, Lys, Leu, Met, Asn, Pro, Gln, Arg, Ser, Thr, Val, Trp, Tyr

SEQ ID NO: 66 - protein, 212 aa, Homo sapiens
  MISC_FEATURE (54)..(54): Xaa = Deleted Amino Acid
AVPSPPPASPRSQYNFIADVVEKTAPAVVYIEILDRHPFLGREVPISNGSGFVXAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 67 - protein, 212 aa, Homo sapiens
  MISC_FEATURE (65)..(65): Xaa = His, Lys, Arg, Phe, Tyr, Trp
AVPSPPPASPRSQYNFIADVVEKTAPAVVYIEILDRHPFLGREVPISNGSGFVVAADGLIVTNAXVVADRRRVRVRXLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 68 - protein, 212 aa, Homo sapiens
  MISC_FEATURE (65)..(65): Xaa = Ala, Asp, Asn, Cys, Glu, Gln, Gly, Ile, Leu, Met, Pro, Ser, Thr, Val
AVPSPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFXVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 69 - protein, 212 aa, Homo sapiens
  MISC_FEATURE (65)..(65): Xaa = His, Lys, Arg, Phe, Tyr, Trp
AVPSPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPXVNLDGEVIGVNTMKVTAGISFAIPSDRLREFLHRG

SEQ ID NO: 70 - protein, 206 aa, Homo sapiens
PPPASPRSQYNFIADVVEKTAPAVVYIEILDRHPFLGREVPISNGSGFVVAADGLIVTNAHVVADRRRVRVRLLSGDTYEAVVTAVDPVADIATLRIQTKEPLPTLPLGRSADVRQGEFVVAMGSPFALQNTITSGIVSSAQRPARDLGLPQTNVEYIQTDAAIDFGNSGGPLVNLDGEVIGVNTMKVTAGISFAIPSDRLREFLH

SEQ ID NO: 71 - protein, 210 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLH

SEQ ID NO: 72 - protein, 212 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVXAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 73 - protein, 212 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVVAADGLIVTNAXVVADRRRVRVRXLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 74 - protein, 212 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFXVAMGSPFALQNTITSGIVSSAQRPARDLGLPQTNVEYIQTDAAIDFGNXGGPLVNLDGEVIGVNTMKVTAGISFAIPSDRLREFLHRG

SEQ ID NO: 75 - protein, 212 aa, Homo sapiens
  MISC_FEATURE (1)..(4): Xaa = Cleaved Amino Acids
XXXXPPPASPRSQYNFIADVVEKTAPAVVYIEILDRHPFLGREVPISNGSGFVVAADGLIVTNAXVVADRRRVRVRLLSGDTYEAVVTAVDPVAXIATLRIQTKEPLPTLPLGRSADVRQGEFVVAMGSPFALQNTITSGIVSSAQRPARDLGLPQTNVEYIQTDAAIDFGNXGGPXVNLDGEVIGVNTMKVTAGISFAIPSDRLREFLHRG

SEQ ID NO: 76 - protein, 100 aa, Homo sapiens
RRYIGVMMLTLSPSILAELQLREPSFPDVQHGVLIHKVILGSPAHRAGLRPGDVILAIGEQMVQNAEDVYEAVRTQSQLAVQIRRGRETLTLYVTPEVTE

SEQ ID NO: 77 - protein, 4 aa, Homo sapiens
AVPS

SEQ ID NO: 78 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 79 - protein, 604 aa, Homo sapiens
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSDLPVEEQLRRLQEERTCKVCMDKEVSIVFIPCGHLVVCKDCAPSLRKCPICRSTIKGTVRTFLS

SEQ ID NO: 80 - protein, 10 aa, Homo sapiens
EQKLISEEDL

SEQ ID NO: 81 - DNA, 26 bp, Homo sapiens
aatctagaatggctgcgccgagggcg

SEQ ID NO: 82 - DNA, 61 bp, Homo sapiens
aaggtacctacaggtcctcctctgagatcagcttctgctcttctgtgacctcaggggtcac

SEQ ID NO: 83 - DNA, 32 bp, Homo sapiens
aatctagaatggccgtccctagcccgccgccc

(19) United States
(12) Patent Application Publication
(10) Pub. No.: US 2005/0233411 A9
(43) Pub. Date: Oct. 20, 2005

(54) Compositions and methods for cleaving IAP

(76) Inventors: Chunying Du, Leawood, KS (US); Qiheng Yang, Kansas City, KS (US)

Correspondence Address: POLSINELLI SHALTON WELTE SUELTHAUS P.C., 700 W. 47TH STREET, SUITE 1000, KANSAS CITY, MO, 64112-1802, US

(21) Appl. No.: 10/730,476

(22) Filed: Dec. 8, 2003

Related U.S. Application Data
(60) Provisional application No. 60/445,508, filed on Feb. 7, 2003.

(51) Int. Cl. C12P 21/06

(52) U.S. Cl. 435/68.1

(57) ABSTRACT

The present invention relates to compositions and methods for making and using Omi-related and IAP-cleaving nucleotide sequences, mutant nucleotide sequences, and polypeptide sequences expressed therefrom, including both biologically active and inactive molecules. The present invention relates to cleaving IAP using an Omi polypeptide.

This application is a non-provisional patent application based on U.S. Provisional Patent Application Serial No. 60/445,508, filed Feb. 7, 2003.

### FIELD OF INVENTION

The present invention relates to methods and compositions, which facilitate caspase activity and regulate apoptosis. In particular, the present invention relates to Omi nucleic acid sequences, and amino acid sequences expressed therefrom, which cleave an Inhibitor of Apoptosis (IAP) molecule and release caspase.

### BACKGROUND OF INVENTION

“Apoptosis” is the programmed death of cells in various tissues at specific times during embryogenesis and metamorphosis, or during cell turnover in adult tissues. For example, approximately 12% of the cells formed during the development of an adult hermaphroditic Caenorhabditis elegans (C. elegans) are destined to die because of a genetically controlled suicide program. If genes functioning in this system are inactivated by mutation, cells that normally die will survive. Apoptosis is a cell death process which occurs during development and aging of animals. Besides genetically controlled suicide, apoptosis can be induced by cytotoxic lymphocytes (CTL), anti-cancer drugs, γ- or UV-irradiation, a group of cytokines called death factors, and deprivation of survival factors.

One of the key regulatory steps for apoptosis is the activation of caspases, which facilitate apoptosis. Activated caspases cause the characteristic morphological changes associated with apoptotic cells. These morphological changes include chromatin condensation, DNA fragmentation into nucleosomal fragments, nuclear membrane break down, externalization of phosphotidylserine, and formation of apoptotic bodies that are readily phagocytosed. As such, activated caspases promote apoptosis, which resultingly causes cell death.

An example of an apoptotic caspase activation cascade is triggered by cytochrome c, a protein that normally functions in the electron transfer chain in mitochondria. In living cells, holocytochrome c is located exclusively in the intermembrane space of the mitochondria, and is, therefore, sequestered away from its deadly cytosolic partner, Apaf-1. Upon receiving apoptotic stimuli, such as serum deprivation, activation of cell surface death receptors, or excessive damage of DNA, the outer membrane of mitochondria becomes permeable to cytochrome c. Once released to the cytosol, cytochrome c binds to Apaf-1 with 2:1 stoichiometry and forms an oligomeric Apaf-1/cytochrome c complex in the presence of dATP or ATP. This oligomerized Apaf-1/cytochrome c complex then recruits and activates the apical caspase of this pathway, procaspase-9. Caspase-9, in turn, activates downstream caspases, such as caspase-3, -6, and -7 that constitute the major caspase activity in an apoptotic cell.

It is known that apoptosis is executed mainly by proteolytic activation of procaspases (zymogens), a group of intracellular cysteine proteases that cleave their substrates after the aspartic acid residue. The cleaved and, thus, activated caspases catalytically degrade some intracellucular molecules and execute cell death. The initial proteolytic cleavage of zymogens is through the extrinsic cell surface pathway through activation of the Tumor Necrosis Factor (TNF) family of receptors, or from the intrinsic route via the release of a group of apoptotic proteins from the mitochondria to cytoplasm. Thus, apoptosis is mediated by a family of proteases called caspases that are activated by converting from the inactive precursor (zymogen) or procaspase to the active caspase.

Thirteen members of the human caspase family have been identified. Some of the family members are involved in apoptosis, and these can be divided into two subgroups. The first group consists of caspase 8, caspase 9, and caspase 10, which contain a long prodomain at the N-terminus, and function as initiators of the cell death process. The second group contains caspase 3, caspase 6, and caspase 7, which have a short prodomain and work as effectors, cleaving various death substrates that ultimately cause the morphological and biochemical changes seen in apoptotic cells.

There are eight known IAP protein molecules in mammals. Of particular interest are the cIAP1, cIAP2, and XIAP molecules, which contain a RING zinc-binding motif at the C-terminus, which functions to modify proteins post-translationally through ubiquitination. Thus, not only do cIAP1, cIAP2, and XIAP bind and inhibit caspase, they also can ubiquitinize and, ultimately, degrade caspase. Ubiquitin-ligase enzymes, such as IAP, add ubiquitins to proteins carrying particular degradation signals. Thereafter, additional ubiquitins are attached to the original ubiquitin to form a poly-ubiquitin chain. This is recognized by proteosomes, which then cut the targeted proteins into fragments. Conjugation of ubiquitin (Ub) to substrate proteins requires three enzymes: a Ub-activating enzyme (E1), a Ub-conjugating enzyme (E2), and a Ub ligase (E3). The RING domain severs proteosome-mediated protein degradation for IAPs, the caspase partners of IAPs, and Smac. As such, if the RING domain of IAP is cleaved, IAP no longer ubiquitinizes a target such as caspase.

The activated caspases can be regulated by the IAP proteins. IAPs block caspase activity by direct binding through Baculovirus IAP Repeat (BIR) domains, which comprise a portion of the IAP. The BIR domains are composed of approximately 70-amino acid residues. It is known that BIR2 and BIR3 and XLAP are the domains that bind and inhibit activated caspases. The BIR3 domain of XIAP specifically inhibits activated caspase 9. The BIR2 domain of XIAP inhibits activated caspase 3. As such, it is desired to have molecules that can be used to promote or block BIR interaction with caspase. It is also desired to have compositions, or methods which can cleave BIR from the remainder of the IAP molecule.

Related to the caspase activation cascade is the protein known as Smac. The Smac protein is a novel factor that promotes cytochrome c/Apaf-1-dependent caspase activation. Like cytochrome c, this protein is normally located in mitochondria and released into cytosol when cells undergo apoptosis. The acronym Smac stands for the second mitochondria-derived activator of caspase, after cytochrome c. The addition of Smac to cytosolic extracts causes robust caspase activation in these extracts with the addition of dATP. Smac promotes caspase activation by out-competing IAP for caspase inhibitory binding sites. Thus, Smac is a protein that promotes caspase activation and, ultimately, apoptosis. It is desired, however, to have other proteins and polypeptides, which can be used in association with caspase activation and apoptosis. It is especially desired to have a molecule that not only prevents binding of IAP to caspase, but that enzymatically degrades IAP.

Smac/DIABLO and Omi/HtrA2 are two molecules identified as antagonists of IAPs. These molecules can reactivate the IAP-inhibited caspases. Smac and Omi are nuclear-encoded mitochondria proteins. It is known that after being synthesized in the cytoplasm, Smac and Omi are quickly imported into the mitochondria by the N-terminal mitochondria targeting peptides. The cleavage of peptides attached to Smac or Omi, inside the mitochondria, generates active Smac and Omi molecules with a new apoptogenic N-terminus, named the IAP binding motif. This motif consists of a short stretch of hydrophobic amino acids AVPI and AVPS in Smac and Omi, respectively. It has been observed that in the cytosol, the IAP binding motifs of Smac and Omi antagonize IAPs' inhibition of caspases by competitively binding to the BIR2 and BIR3 domains of IAPs so the BIR domain-bound caspases are released and reactivated. As such, it has been determined that Smac and Omi competively bind LAP to prevent IAP inhibition of caspase.

A conserved stretch of IAP binding motif is present in the Drosophila apoptotic proteins Reaper, Grim, Hid, and recently reported Sickle, and is the antagonist of the IAPs in Drosophila. The peptides generated from the N-terminus of these Drosophila proteins can also antagonize mammalian IAPs, indicating an evolutionarily conserved mechanism in regulating apoptosis.

It has been known that serine protease at position 306 (S306) of Omi causes degradation of various proteins, including β-casein; however, such protease activity has not been associated previously with IAP. In fact, it is desired to have a protease that not only binds to IAP, but proteolytically degrades IAP. It is further desired to have compositions and methods for inhibiting or degrading IAPs and promoting apoptosis. In particular, it is desired to have compositions and methods for cleaving IAPs. It is further desired to have small molecules and other compositions that can be used to cleave IAP, or, conversely, to prevent degradation of IAP. It is further desired to have kits and tools for detecting molecules that cleave IAPs. It is especially desired to have methods and compositions related to the use of the Omi protein.

### SUMMARY OF INVENTION

The present invention relates to methods for cleaving IAP, both in vivo and in vitro, wherein an Omi family polypeptide or polynucleotide sequence is used to promote the cleavage of IAP. In particular, the present invention relates to an active Omi polypeptide that cleaves LAP and renders it non-functional. The Omi family polypeptides will include Omi wild type (WT) sequences and mutant versions, which cleave IAP. Among the available mutant Omi family polypeptides are OmiΔPDZ and OmiΔAVPS. Additionally, an Omi catalytic triad may be used to cleave LAP. Related to the polypeptides are nucleic acid sequences, which express the polypeptides. Various methods can be used to deliver the Omi nucleic acid sequences, or polynucleotides, or the Omi polypeptides. Additionally, mutant versions can be used to block or inhibit Omi WT from cleaving IAP. In particular, an AVPS small molecule can be developed, which inhibits the binding of Omi WT, for example, to an IAP.

The present invention relates to regulators of enzymes associated with apoptosis. The invention provides methods and compositions relating to polypeptide regulators (activators and inhibitors) of enzymes involved in cellular apoptosis, particularly caspases. In a particular aspect, the invention provides polypeptide and polynucleotide sequences which cleave IAP. These polypeptides and polynucleotides offer a variety of diagnostic and therapeutic applications involving detecting or modulating expression or the function of activators, caspases, and genes or transcripts encoding such activators. Genetic and immunogenic probes specific for activators of caspases are also provided.

Since undesirable activation or inactivation of apoptosis has been associated with many human diseases, such as cancer, autoimmune diseases and neurodegenerative diseases, the disclosed caspase regulatory polypeptides and polynucleotides provide both drug targets and regulators to promote or inhibit apoptosis. In particular, Omi provides a molecule for cleaving IAP and promoting apoptosis.

The present invention specifically relates to a method for cleaving IAP and causing caspase activation, where, for example, an IAP is bound to a caspase. The method is initiated by contacting an IAP bound to a caspase with an amount of an Omi family polypeptide, whereby upon contact, Omi will cleave IP and release the caspase from IAP. LAP is found in eukaryotic cells. Specific IAPs cleaved by Omi polypeptides include cIP1, cIAP2, XIAP, Livin α, Livin β, and DIAP1. Suitable Omi polypeptides are listed in SEQ ID NOs. 44, 45, 48, 49, 52-57, 60-63, 66-75, and include homologs and degenerate variants thereof.

IAP that is cleaved by an Omi polypeptide is resultingly BIR2 deficient. IAP is derived from cells selected from the group consisting of mammalian, reptile, aves, and amphibian cells. The method is conducted in vitro or in vivo. The present method can also be used for preventing IAP ubiquitination of caspase thus causing caspase activation when IAP is bound to a caspase. The method includes contacting an LAP bound to a caspase with an amount of an Omi polypeptide, whereby upon contact, Omi will cleave LAP and release the caspase from IAP. The method ultimately promotes apoptosis and causes caspase activation when LAP is bound to a caspase.

Omi polypeptides and, more particularly, active Omi polypeptide family members, are expressed by nucleic acid sequence molecules or polynucleotides that include SEQ ID NOs. 1-3, 6-8, 11-19, 22-27, and 30-39. SEQ ID NOs. 1-40 relate to Omi or Omi family member nucleic acid sequences, both active and inactive. SEQ ID NOs. 44-77 relate to Omi family member polypeptides, both active and inactive.

The polypeptides for cleaving IAP will have a protease domain shown in SEQ ID NOs. 44, 45, 48, 49, 52-57, 60-63, and 66-75. Homologous sequences to these protease domains are also available for use. Available polypeptides include Omi, OmiΔPDZ, Omi protease, Omi catalytic triad, and homologs thereof A specific polypeptide having increased protease activity is OmiΔPDZ, including SEQ ID NOs. 48, 49, 56, 57, 60, 61, 62, 63, and 66-75. The polypeptide that binds a BIR site on IAP is the AVPS peptide sequence of SEQ ID NO. 77.

The present invention also relates to a polypeptide molecule for cleaving IAP comprising an amino acid sequence as set forth in the formula C1ₙ₁-R1-C2ₙ₂-R2-C3ₙ₃-R3-C4_{n4 serine; R}2 is an amino acid residue selected from a group consisting of a charged amino acid residue and an aromatic amino acid residue; R3 is an amino acid residue selected from a group consisting of a charged amino acid residue and a polar amino acid residue. R1, R2 and R3 form a catalytic triad for cleavage of LAP. The R2 residue, in the alternative, can be an amino acid residue selected from a group consisting of histidine, lysine, arginine, phenylalanine, tyrosine, and tryptophan. The R3 residue, in the alternative, can be an amino acid residue selected from a group consisting of aspartic acid, glutamic acid, lysine, histidine, and arginine. C1ₙ₁, C2ₙ₂, C3ₙ₃, and C4ₙ₄ are polypeptide chains, with n1 a number between 10 and 100 residues, n2 a number between 10 and 100 residues, n3 a number between 10 and 150 residues, and, n4 a number between 10 and 200 residues. The C1ₙ₁ chain is the N-terminal and has an AVPS motif sequence that operably couples to IAP. The C4ₙ₄ chain is the C-terminal and has a hinge sequence and PDZ domain. As mentioned, the PDZ domain can be removed or mutated.

A polypeptide molecule for cleaving LAP is contemplated, wherein a catalytic triad for cleavage of IAP is formed from amino acid residues serine 306, histidine 198, and glutamic acid 228. The number represents positions in the WT polypeptide. It is contemplated that the triad can be manipulated or used in such a way as to be part of a small molecule for use in cleaving IAP. The triad will include the polypeptide chains which have likely been cleaved to produce a short chain. The triad also serves as a model for use in combinatorial chemistry or polypeptide identification. Both models would ultimately be used in cleaving IAP. An example of a suitable use includes operably enclosing the molecule in a liposome in an aqueous medium, with the available liposomes including unilamellar liposomes and multilamellar liposomes. The liposomes will include a plurality of antibodies on the surface of the liposomes that operably couple the liposomes to a plurality of antigens on a cell membrane of a host cell. The molecule for cleaving LAP enters the cytoplasm of the host cell and kills the host cell. Available host cells include eukaryotic cells and prokaryotic cells, with the eukaryotic cells including animal cells, plant cells, and microbial cells. The microbial cells include bacterial cells, fungal cells, microalgae cells, and protozoa cells. The animal cells include vertebrate cells and invertebrate cells, with the vertebrate cells including amphibian cells, reptilian cells, rodent cells, mammalian cells, and nonhuman primate cells. Also, human cells may be used. The polypeptide can also be delivered via other methods.

An expression vector may be made that includes a polynucleotide that expresses a molecule for cleaving IAP. Available expression vectors include plasmids and episomes. Also, a replicating virus may be used.

Resultant transfected mammalian cells are also part of the present invention. Mammalian cells are transfected with the expression vector. The transfected cell will be activated and cause expression of the transfected polynucleotide to produce an IAP-cleaving molecule, specifically an Omi family polypeptide. The polypeptide will likely cleave IAP found in the cell. Promoters for controlling transcription and the quantity of production of the IAP-cleaving molecules are part of the vector transfected into the cells. The expression vector can be autonomously replicating.

A mammalian recombinant cell produced by a plurality of recombinant DNA techniques, whereby the recombinant cell produces an IAP-cleaving molecule is part of the present invention. A method for producing an IAP-cleaving molecule can be practiced. The method includes culturing mammalian cells previously mentioned.

A pharmaceutical composition for the treatment of a hyperproliferative disorder in a mammal can be used. It includes a pharmacologically acceptable carrier and a therapeutically effective amount of the IP-cleaving molecule. The molecule is generally a liposome. The pharmaceutical composition can be used to treat a hyperproliferative disorder, such as cancer.

Various Omi compositions can be administered to a vertebrate animal by intravenous, topical, oral, subcutaneous, intrathecal, and intramuscular routes. As can be expected, there are a variety of methods available for delivering an Omi family polynucleotide or polypeptide.

It has been determined that the IAP binding motif (AVPS) and the endopeptidase domain are typically required for Omi's pro-apoptotic function. Also, the serine protease functionality of Omi can override the pro-apoptotic activity mediated by Omi's IAP-binding-motif. Omi molecules which are IAP binding motif deficient possess pro-apoptotic activity when over-expressed in cells. The monomeric mutant of Omi bearing the AVPS motif, but deficient in its serine protease activity, is unable to induce cell death. Therefore, the serine protease activity of Omi is likely indispensable for its pro-apoptotic function.

Use of Omi is advantageous because not only does it bind to IAP, but it cleaves IAP. Thus, Omi can permanently inactivate IAP. Omi can be mutanized to make smaller molecules, with such molecules having use in therapeutic applications. Omi polynucleotides and polypeptides may be used, as well as vectors, transfected cells, and small molecules.

### BRIEF DESCRIPTION OF THE DRAWINGS

FIG. 1 relates to Omi cleavage of IAP proteins, different IAP proteins were incubated in the absence or presence of various amounts of Omi WT or the protease dead mutant Omi SA;

FIG. 1A shows the cleavage products of cIAP1, cIAP2, XIAP or DIAP1 (50 nM each) incubated with increasing amounts of Omi WT (0-25 nM), the cleavage products were detected by silver staining after being resolved on the 7.5-20% linear gradient gel;

FIG. 1B shows the results of Livin α or Livin β (50 nM each) incubation with increasing amounts of Omi WT (0-25 nM); the samples were resolved by 13.5% SDS-PAGE, the cleavage products were detected by Western blotting with an antibody against Livin, since Omi co-migrated with Livin in the gel and this could interfere with the identification of Livin cleavage by silver staining;

FIG. 1C shows Survivin (25 nM) incubated with excessive amounts of Omi WT (150 nM), the samples were separated on 13.5% gel and immunoblotted with an antibody against Survivin;

FIG. 1D shows cIAP1 (50 nM) incubated with 150 nM of Omi WT (Lane 2) or Omi SA (Lane 3), the cleavage products were separated on 10% gel and detected by Western blotting with a HRP-conjugated antibody against GST since the cIAP1 was a GST fusion protein;

FIG. 1E shows cIAP1 (50 nM) incubated with 2.5 nM of Omi WT (Lane 2) or 50 nMOmi SA (Lane 3), the cleavage products were separated on 10% gel and detected by Western blotting with a HRP-conjugated antibody against GST since the cIAP1 was a GST fusion protein;

FIG. 2 shows Omi/HtrA2 Cleavage of cIAP1 and the relation to the AVPS IAP binding motif;

FIG. 2A is a schematic representation of wild-type and mutant forms of Omi, the N-terminal solid area represents the AVPS IAP binding motif, the serine protease domain is located in the central region of the molecule, the catalytic residue S306 is also indicated, the C-terminal striped region represents the PDZ domain, the unshaded region represents the hinge;

FIG. 2B shows the cleavage of cIAP1 by various Omi proteins; 50 nM of cIAP was incubated with 2.5 nM of Omi WT (lane 2), varying amounts of OmiΔ8 mutant (lanes 3-7) or OmiΔPDZ mutant (lanes 8-12) in a final volume of 50 μl PBST, the asterisk (*) in panel B indicates a cleavage product produced exclusively by OmiΔPDZ proteolysis of cIAP1;

FIG. 2C shows the results of the GST fusion form of full-length cIAP1 (50 nM) incubated with 100 nM of Omi WT and Omi mutants for 20 minutes at 4° C. in 50 μl of PBST;

FIG. 2D shows the cleavage of β-casein by various Omi proteins; 200 nM of β-casein which was incubated with 2.5 nM of Omi WT (lane 2), varying amounts of OmiΔ8 mutant (lanes 3-7) or OmiΔPDZ mutant (lanes 8-12) in a final volume of 50 μl PBST, the asterisk (*) in panel B indicates a cleavage product produced exclusively by OmiΔPDZ proteolysis of β-casein;

FIG. 3 shows cIAP1 cleavage by Omi/HtrA2 and how cleavage reduces cIAP 1's caspase inhibitory activity;

FIG. 3A shows the results of incubating cIAP1 protein (400 nM) with varying amounts of Smac, Omi WT or Omi SA;

FIG. 3B shows that Omi does not cleave caspase-3 and caspase-9, about 250 ng of either recombinant caspase-3 (lane 4) or caspase-9 (lane 6) was incubated with 50 ng of Omi, Omi cleavage of β-casein was included as a positive control (lane 2), all of the samples were run on the same gel, the splitting of the gel into two parts in this figure presentation was for the convenience of sample labeling, the two parts, therefore, shared the molecular weight marker;

FIG. 4 shows that cIAP1 cleavage by Omi/HtrA2 attenuates its Ub ligase activity on caspase substrates;

FIG. 4A demonstrates the establishment of an in vitro assay for cIAP1 Ub ligase activity using caspase-3 and caspase-9 as the substrates, about 400 nM caspase-3 or caspase-9 was incubated with 200 nM cIAP1 for 2 hours at 30° C. in a 20-μl final reaction volume, this final reaction volume contained 100 nM ubiquitin activating enzyme, 400 nM ubiquitin conjugating enzyme Ubc H6 (E2), 20 μM ubiquitin, 2 mM Mg-ATP, 40 mM Tris-HCl (pH 7.5), and 50 mM NaCl, the ubiquitination of both caspase substrates was analyzed by Western blotting with an antibody against caspase-3 (lanes 1 and 2) or caspase-9 (lanes 3 and 4), both caspase samples are a mixture of the pro-form and the active form as indicated in the figure, the asterisk (*) indicates the mono-ubiquitinated (Ub)₁ active caspase-9, the poly-unbiquitinated caspase-3 and -9 are denoted by (Ub)n;

FIGS. 4B and C show assay for the Ub ligase activity of cIAP1 before and after cleavage, the substrates caspase-3 (400 nM, Panel B) and caspase-9 (400 nM, Panel C) were incubated with varying concentrations of either full length or Omi-cleaved cIAP1 (25-150 nM) in a 20-μl reaction volume under the same assay conditions as described in panel A of this figure, the ubiquitination on caspase substrates was subsequently checked by immunoblotting, using either an antibody against caspase-3 (Panel B) or caspase-9 (Panel C);

FIG. 5 shows mapping of Omi/HtrA2 cleavage sites on cIAP1;

FIG. 5A shows the results of incubating about 5 μg of full-length cIAP1 (GST-fused) with 0.4 μg of Omi WT, the cleaved cIAP1 sample, together with Omi (lane 2), the full-length cIAP1 alone (lane 1), and Omi alone (lane 3) were subjected to electrophoresis on a 7.5-20% linear gradient gel, four cleavage polypeptide fragments (panel A, F1-F4) were generated, 10 pmol of each fragment was excised and subjected to N-terminal sequencing by the Edman Degradation method, the two 30 kDa polypeptides in lane 2 are GST as determined by N-terminal sequencing, several degraded polypeptide bands are already in the full-length cIAP1 preparation, such as that labeled with an asterisk (*), amino acid sequencing confirmed that this band was a fragment of cIAP1 starting from Serine 147, and identical to the band appearing in the OmiWT-treated sample (labeled with an arrow plus an asterisk);

FIG. 5B shows a map of Omi cleavage sites on human cIAP1, the cIAP1 is labeled with the three mapped and unmapped sites, the three underlined amino acid sequences were the amino terminal sequences (determined by Edman Degradation) of the cleaved cIAP1 fragments F1/F2, F3 and F4, respectively, Omi cleaves cIAP1 after the residue Thr4, Asn133, and Leu161 as denoted by the three arrows, both polypeptide fragments F1 and F2 start with the amino acid sequence ASQRLFPG, F6 starts with SFAHSLSP, and F5 with NSRAVEDI;

FIG. 6 shows that Omi cleaves cIAP1 in cells, and this cleavage promotes caspase activation in etoposide-induced cell death;

FIG. 6A shows results of human histiocytic lymphoma U937 cells that were left untreated or treated with 100 μM etoposide or 2 μM staurosporine; a filter was probed with an antibody against cIAP1, the arrow indicates the full-length cIAP1 molecule, and the asterisk (*) indicates an unrelated polypeptide band;

FIG. 6B is a schematic representation of Omi expression constructs, the upper two diagrams represent the full-length Omi constructs with the mitochondrial targeting sequences (MTS) at the N-terminal part of the molecule, the lower two diagrams represent the cytosolic form of Omi with the IAP binding motif AVPS at their N-termini, the central region represents the protease domain with either a wild-type protease (S306) or an inactivated protease (A306), the hatched region represents the PDZ domain, the C-terminal end represents the engineered c-Myc tag in the constructs;

FIG. 6C shows that full-length Omi WT, but not the protease dead mutant Omi SA, cleaves cIAP1 in cells during etoposide-induced cell death, the arrowhead indicates the cleavage product of cIAP1 upon etoposide treatment, the asterisk (*) indicates the polypeptides unrelated to this etoposide treatment;

FIG. 6D shows that the cytosolic form of Omi WT, but not the protease dead Omi SA, cleaves cIAP1 in cultured cells, HEK 293 cells were transfected with 1.5 μg of both the C-terminal c-Myc tagged cytosolic form of Omi, either wild-type (AVPS Omi WT) or the protease dead mutant (AVPS Omi SA), and N-terminal FLAG tagged full-length cIAP1 expression constructs, after transfection for 24 hours, the cells were treated with 100 μM etoposide and harvested at different time intervals of 24 hours and 48 hours, the arrowhead in the middle and lower panels indicates the cleaved fragment of caspase-8 and -3, respectively;

FIG. 7A shows wild-type or mutant cIAP1 proteins at 200 nM that were preincubated with Omi and assayed for caspase inhibitory activity in HeLa S 100 extracts suplemented with dATP and cytochrome c, the caspase-3 cleavage activity, detected on a Phosphorimager (top panel), with cleavage of cIAP1 detected on the same filter by an anti-GST antibody (bottom panel),

FIG. 7B shows that Omi was detected with a polyclonal antibody (middle panel) so that both the endogenous (lower band) and exogenously expressed (upper band) Myc-tagged Omi were detected, Immuno-blotting for Actin was to show equal sample loadings (bottom panel);

FIG. 7C the DEVD activity for the samples in 7B were plotted to represent the DEVD activity for the same numbered samples in 7B, the curve that lies on the X-axis (Δ) is the DEVD activity for samples in lanes 1-4, 8, and 10 in FIG. 7B;

FIG. 8A siRNA oligonucleotides against Omi (si-Omi) were transfected twice into HeLa cells with Luciferase GL2 siRNA duplex as control (Ctrl), ten μg of protein per sample were subjected to immunoblotting for endogenous Omi and cIAP1, Immunoblotting for Actin was done to show equal sample loadings; and,

FIG. 8B DEVD activity assay for the samples in 8A, the number next to each curve represents the DEVD activity for the same numbered samples in 8A.

### DETAILED DESCRIPTION

The present invention relates to methods and compositions for cleaving IAP and, resultingly, promoting caspase activation. The activation of caspase will lead to apoptosis in a cell. The composition for cleaving LAP is an Omi protein, polypeptide, or amino acid sequence. As such, the present invention relates to methods for using Omi and related polypeptides for cleaving IAP. The present invention also relates to the Omi gene, polynucleotides, and related nucleic acid sequence molecules. The Omi polypeptides and related nucleic acid sequences can be used as part of various methods to promote or prevent apoptosis, in particular, to enzymatically cleave IAP.

More particularly, the present invention relates to genes, polynucleotides, or nucleic acid sequences that encode the serine protease, Omi, and related polypeptides. Purified and isolated preparations of Omi, recombinant preparations of Omi, and a variety of other Omi polypeptides can be used herewith. Polypeptides expressed from the Omi nucleic acid sequences and related nucleic acid sequences are considered part of the present invention. The Omi polypeptide can be a full length sequence or a fragment of the full length of the mature Omi wild-type (WT) polypeptide of SEQ ID NO. 44. The present invention further relates to mutant nucleic acid and amino acid sequences known as OmiΔPDZ, OmiΔAVPS, Omi serine protease catalytic triad, and Omi serine protease. These various polypeptides and nucleic acid sequences are known as the Omi family members. Related to these constituents are mutants, degenerate sequences, and homologs. The nucleic acid and amino acid sequences referenced herein are disclosed in the sequence listing section.

The Omi WT gene is SEQ ID NO. 1. The gene is comprised of 975 nucleic acids and is isolated from Homo sapiens (humans). It should be noted that there are wild-type variations of the Omi gene disclosed in the literature. Such sequences may be of fewer or more nucleic acids. SEQ ID NO. 1 is a mature Omi sequence and, as such, some nucleic acids may be excluded when compared to other disclosed Omi sequences. Regardless, the wild-type expresses a serine protease that includes a catalytic triad that cleaves IAP, and includes a PDZ domain and an AVPS domain. The PDZ domain encodes a polypeptide that regulates the activity of the Omi polypeptide. The AVPS domain encodes a peptide sequence that binds Omi to IAP. The Omi gene or sequence includes three codons that encode an amino acid catalytic triad that cleaves IAP. The Omi gene is found in a variety of eukaryotic organisms including mammals, in particular, humans and primates. Omi is also known as HTRA2 or PRSS25. The Omi or HTRA2 gene is located at 2p12 on the human chromosome. The Omi gene is associated with the mitochondria, with the expressed polypeptide released into the cytosol following apoptotic stimulus. As such, the Omi gene expresses a stress-regulated endoprotease.

As mentioned, the Omi WT nucleic acid sequence includes three codons which, when expressed, form a catalytic triad in the protease molecule. The nucleic acids, which form the codons, are at positions 193-195, 283-285, and 517-519 on the mature Omi WT nucleic acid sequence, SEQ ID NO. 1. The PDZ domain is located between nucleic acids 675 and 975 of SEQ ID NO. 1. The AVPS nucleic acids are nucleotides 1-12 of SEQ ID NO. 1. The hinge sequence is located between nucleic acids 636 and 675. As will be shown, these sequences can be removed or mutated. It is important to note that the Omi WT nucleic acid sequence should encode a serine at position 306 (S306) of the active protease polypeptide expressed by the wild-type version of Omi. Thus, the expressed polypeptide includes a catalytic triad, which includes S306. The mature polypeptide, SEQ ID NO. 44, shows the serine at position 173. This is a mature Omi polypeptide that excludes 133 amino acid residues located prior to the AVPS sequence. As such, S306 and S 173 are interchangeable for purposes of this application. Either way, the Omi polypeptide enzymatically cleaves IAP.

The Omi nucleic acid sequence expresses a polypeptide that specifically cleaves certain target proteins. In particular, IAP and Livin are cleaved by Omi. Other targets, such as Survivin, are not cleaved, meaning, Omi cleaves with specificity.

As will be shown, variations of the Omi WT nucleic acid sequence can be used. Suitable homologous sequences will express the catalytic triad, as well as the PDZ and AVPS domains. As such, nucleic acid sequences which are homologous to the listed sequences are available for use as long as the homologous sequence expresses a polypeptide homologous to, or having the same functionality as, one of the polypeptides listed herein. It is important that the homologs express a polypeptide that has the same activity. In particular, the expressed polypeptide will preferably catalytically degrade IAP. Sequences having homologous catalytic triads, while the rest of the polypeptide in the sequences are not homologous, are available for use. Other available homologous sequences should be at least 50% homologous to Omi WT, with the triad sequences homologous and capable of expression. A sequence that is 64% homologous is available to use; such sequence eliminates the PDZ, hinge, and AVPS domains, but is homologous elsewhere, including the triad. Degenerate variants are also available for use. Importantly, the homologous sequences, or degenerate variants, should include a catalytic triad which codes for a serine at position 306, or the equivalent. Various Omi family member sequences can be used for a variety of methods and applications. Fragments of the entire Omi WT gene can be used. Selected fragments can include the catalytic triad.

Nucleic acid substitutions of the wild-type Omi nucleic acid sequence can occur at positions 193-195, 283-285, and 517-519 of SEQ ID NO. 1. Nucleic acids at positions 517-519 relate to a codon that encodes a serine, nucleic acids 193-195 encode a histidine, and nucleic acids 283-285 encode an aspartanine. If the serine is converted to a different amino acid, the expressed polypeptide will not enzymatically degrade IAP. Substitutions, however, can be made in any of the three codons, with the substitutions dependent upon the desired use of the polypeptide. The substitutions can inactivate the enzyme or can be such that enzymatic activity remains the same. The substitutions can be such that the same amino acid residue is expressed, or a different residue having the same functionality is expressed. As stated, the three codons code for the amino acid residues that form the catalytic triad. The substitutions are designed to allow for or eliminate protease activity. SEQ ID NOs. 2 and 3 disclose Omi WT nucleic acid sequences where substitutions have occurred. Members of the catalytic triad are substituted, but the resultant protease activity remains the same. In SEQ ID NOs. 4 and 5, the substitutions are structured so that the catalytic activity in the resultant polypeptide triad is eliminated. The substitutions are illustrated in sequences where the nucleic acid equals n, this is true in all of the sequence listings. The nucleic acids, which are available for substitution, are listed in the sequence listing.

The structure of the triad can be useful for future applications, such as forming small molecules for therapeutic uses. Substitutions where activity remains the same may be well suited for use in small molecule design, whereby the molecule is designed to cleave the IAP. If the enzymatic activity is eliminated, such molecule may also have value in blocking Omi WT.

The Omi WT nucleic acid sequence can be manipulated, whereby the nucleic acid sequence or fragment that codes the PDZ domain is eliminated. When the PDZ domain is eliminated, a more protealytically active polypeptide expressed from the nucleic acid sequence is formed. The PDZ domain regulates the enzymatic activity of the Omi polypeptide. If the PDZ domain is removed, the polypeptide is more enzymatically active, meaning it more readily degrades IAP. The isolated PDZ nucleic acid sequence domain is SEQ ID NO. 40. To eliminate the functionality of the PDZ domain, nucleic acids 676 through 975 are removed, cleaved, or mutated. SEQ ID NOs. 6, 7, and 8 are structured, whereby the PDZ domain is eliminated from the Omi WT. In addition to removing the PDZ domain, the catalytic triad can be altered to have substitutions, as shown in SEQ ID NOs. 7 and 8. As such, the nucleic acids which encode PDZ can be removed or mutated. Substitutions can be made in the catalytic triad after removal or mutation of the PDZ domain, so that the nucleic acid sequences can encode an inactive catalytic triad.

When the PDZ domain is removed, it is known as an OmiΔPDZ nucleic acid sequence or polypeptide. In some cases, OmiΔPDZ polypeptide is preferred for use because of the increased proteolytic activity. SEQ ID NOs. 6-10 and 14-39 are variations of the OmiΔPDZ nucleic acid sequence. SEQ ID Nos. 9 and 10 have the PDZ domain removed and express an inactive enzyme because of substitutions in the triad.

The Omi WT can be treated so that the nucleic acid sequence that encodes the AVPS domain of the Omi polypeptide is eliminated or mutated. The AVPS tetrapeptide domain is the binding site for the IAP and competes with Smac for binding sites on the IAP. The AVPS nucleic acid sequence is comprised of 12 nucleotides. SEQ ID NO. 41 is the isolated AVPS nucleic acid sequence. Additionally, the AVPS can be used alone to competitively bind with Smac or block Omi from binding to IAP.

When the AVPS domain is removed, the resultant molecule is known as OmiΔAVPS. The catalytic triad can be altered along with the removal or mutation of the AVPS nucleic acid sequence. SEQ ID NOs. 11-13 are the Omi nucleic acid sequence without AVPS. SEQ ID NOs. 12 and 13 are the active Omi nucleic acid sequence with substitutions in the triad.

The Omi WT nucleic acid sequence can be mutated so that the AVPS and PDZ nucleic acid sequence domains are eliminated. This is known as an Omi serine protease nucleic acid sequence, wherein the sequence is without PDZ and AVPS domains. The hinge can also be removed. The Omi protease expresses a polypeptide that includes the triad and associated residue chains. SEQ ID NOs. 14-16 are Omi with the AVPS removed, as well as a mutation of other parts of the sequence resulting in an inactive polypeptide. SEQ ID NO. 14 is the Omi serine protease nucleic acid sequence with AVPS and PDZ removed. Additionally, the catalytic triad can be altered or substituted concurrent with the removal of AVPS & PDZ. SEQ ID NOs. 15 and 16 are active Omi serine protease sequences with substitutions in the triad.

As mentioned, the hinge region can be removed from the Omi WT. The hinge region is comprised of nucleotides 636 through 675 of SEQ ID NO. 1. The hinge region is associated with the PDZ domain, and helps regulate Omi. Typically, the hinge region is removed, or mutated with the PDZ region. SEQ ID NOs. 17-39 are nucleic acid sequences where the hinge region has been removed. SEQ ID NOs. 17-21, are sequences where the hinge and PDZ regions have been removed together. SEQ ID NO. 17 includes no substitutions to the triad and is active. SEQ ID NOs. 18 and 19 include substitutions in the triad and are active. SEQ ID NOs. 20 and 21 have triad substitutions and express inactive enzymes.

The Omi catalytic triad, without the AVPS, PDZ, and hinge domains, is available for use. SEQ ID NOs. 22-39 are variations of the Omi catalytic triad nucleic acid sequence. The AVPS, the hinge, and the PDZ domains are eliminated from the nucleotide sequence, which includes the catalytic triad, shown in SEQ ID NO. 22. The residue chains are also mutated or cleaved to form the triad. As before, the triad can be substituted, with SEQ ID NOs. 23 and 24 shown as substituted and active. The triad includes the three codons, which express the amino acids, which cause cleavage of the IAP. As can be seen, some of the listed catalytic triads form inactive enzymes. The active and inactive triad can be used to form various vectors and small molecules. The triads include additional nucleotides, which express polypeptide chains. The inactive triads are illustrated in SEQ ID NOs. 28-29. As such, polynucleotides which form the residue chains can be removed.

Finally, the Omi WT sequence can be of a short form by removing nucleotides encoding the residue chains. SEQ ID NO. 25 is an active sequence that does not include the hinge, PDZ, and two codons, which form part of the residue chains. The triad in SEQ ID NO. 25 is not substituted; however, SEQ ID NOs. 26 and 27 are short, active and have substitutions in the triad. SEQ ID NOs. 28 and 29 are short inactive enzymes.

SEQ ID NOs. 30-33 have the hinge and PDZ removed. Nucleotides within the sequence are removed, such as nucleotides 160-162. The sequences are active, but have nucleotides, which encode residue chains deleted. SEQ ID NO. 34 has AVPS, the hinge, and PDZ removed along with six nucleotides at the end. SEQ ID NOs. 35, 36, 37, 38, and 39 are the same, but with triad additions or alterations.

Nucleic acid sequences, which include OmiΔPDZ, OmiΔAVPS, protease domains, catalytic triad, and the other above mentioned sequences, are available for use and include homologs, degenerate variants and antisense molecules of the above-mentioned sequences. Alternative sequences, such as homologs, are available for use in enzymatically degrading IAP. As such, the alternative sequences can be used as part of a method to cleave IAP and promote apoptosis. Degenerate variants are well suited for use as alternative sequences for use in cleaving IAP. Sequences homologous to the individual domains may also be used.

Nucleic acid sequences are defined to include DNA and RNA, RNA sequences expressed from the above DNA nucleic acid sequences may be used herewith. The RNA sequences can be isolated and manipulated in the same way as DNA nucleic acid sequences. Also, homologs, degenerate variants, and antisense molecules to the RNA may be used. The above Omi nucleic acid sequences, SEQ ID NOs. 1-41 are known as the Omi nucleic acid sequence family members.

The various nucleic acid sequences mentioned above can be obtained using a variety of different techniques. The wild-type Omi, as well as homologous sequences, can be isolated using standard known techniques, or can be purchased or obtained from a depository. Once the Omi WT nucleic acid sequence is isolated, it can be amplified for use in a variety of applications.

The removal or mutation of the nucleic acid sequences can be achieved using any of a variety of techniques. For example, the point mutation and various deletion mutations of Omi were generated by PCR.

Deletion or substitution mutations can be made to the Omi WT nucleic acid sequence to form SEQ ID NOs. 2-41. Once the Omi WT, mutants, homologous sequences, or any other Omi sequence discussed herein are formed, they can be amplified for placement in a vector, or used as part of a small molecule. The small molecules can be used to cleave IAP or block Omi binding to IAP. The sequences can also be used to identify candidate small molecules. Amplification can occur using standard PCR techniques. As such, the nucleic acids, such as RNA or DNA, encode an Omi polypeptide or variant. This can include double stranded nucleic acids, as well as coding and antisense single strands.

The isolated Omi family member nucleic acid sequences can be placed into various vectors, such as expression vectors, fusion vectors, gene therapy vectors, two-hybrid vectors, reverse two-hybrid vectors, sequencing vectors, and cloning vectors. The vectors can include activator or promoter sequences, as well as markers. An inducible promoter may also be included in the vector. The resultant vector will include an Omi family member nucleic acid sequence and, optionally, a marker or activator. It is preferred to include a promoter.

Selectable marker genes are introduced into vectors by recombinant DNA technological methods, wherein the vector is introduced into a cell. Selectable markers are used to ensure that a targeted nucleic acid sequence has been incorporated into the vector. There are three general categories of selectable marker genes available, including antibiotic resistant marker genes, metabolic/auxotrophic marker genes, and screenable marker genes. Antibiotic resistant marker genes confer the phenotypic trait of resistance to a specific antibiotic. For example, the neomycin phosphotransferase II (NPT II) gene is a selectable marker for resistance to the antibiotics neomycin and kanamycin.

Metabolic or auxotrophic marker genes enable transformed cells to synthesize an essential component, usually an amino acid, which the cells cannot otherwise produce. The cell culture medium is made to intentionally lack the essential component, which cells require for growth. Cells that have successfully incorporated the selectable marker and remainder of the gene construct will produce the essential components intracellularly, and thereby survive and grow in the component-deficient medium. These cells can be selected and regenerated into whole mutant organisms.

Finally, screenable markers, also known as assayable markers, are genes which encode for a protein that can then be readily identified through other laboratory methods. The presence of the protein confirms that transformation has taken place. Examples of screenable markers which are epitope tags are HIS, MYC, HA, HSV, V5, and FLAG. These sequences encode short peptides that create an antigenic determinant (eiptope) that can be recognized by antibodies. Thus, when the DNA sequence of interest is linked with the DNA sequence of the short peptide, the resulting exported protein is now a “tagged” protein. Since antibodies to the peptide tag are readily available commercially, immunoprecipitation or immunopurification of the tagged fusion proteins can be accomplished. The selectable marker for use herewith is preferably selected from the group consisting of the antibiotic resistance marker neo, and the screenable markers LacZ, Fc, DIG, MYC, and FLAG. These markers were selected as some of the most prevalently used markers in the field, and the methods associated with them are well established. Other selectable markers that can be utilized are Bar/Pat, Bla, dhfr, aadA, Hpt, Epsps/AroA, Gox, Bxn, Als, tdc, Badh, ble, and csr1. Inclusion of the markers is optional.

As stated, vectors can be used to deliver an Omi family polynucleotide to a host cell. In gene therapy, the nucleotide sequence for a therapeutic protein, for example Omi, is incorporated into an expression vector which subsequently transfects a target cell. The vector binds to the target cell membrane, with internalization of the therapeutic nucleotide sequence into the cell. The vector's nucleic acid sequence is integrated into the target cell nucleic acid sequence, and the therapeutic protein is expressed. As such, a suitable vector for the present invention is one that can transfect a desired cell and deliver an Omi family nucleotide sequence.

Suitable eukaryotic gene transfer expression vectors are retroviruses, adenoviruses, adeno-associated viruses, and herpes viruses. A preferred vector should be of a size suitable for the addition of an Omi family member nucleotide sequence. The vector typically should transfect mammalian cells. Retroviruses can package up to 5 kb of exogenous nucleic acid material, and can efficiently infect dividing cells via a specific receptor, wherein the exogenous genetic information is integrated into the target cell genome. In the host cell cytoplasm, the reverse transcriptase enzyme carried by the vector converts the RNA into proviral DNA, which is then integrated into the target cell genome, thereby expressing the transgene product.

Adenoviruses are large double-stranded DNA viruses which contain a 36 kb genome that consists of early regulatory proteins encoding genes and a late structural protein gene. Adenoviruses can be grown in high titers of purified recombinant virus (up to 1012 infectious particles/ml), incorporate large amounts of exogenous genetic information, and can broadly infect a wide range of differentiated non-dividing cells in vivo.

Adeno-associated virus (AAV) is a human parvovirus that is a small single-stranded DNA virus that can infect both dividing and non-dividing cells. AAV is relatively non-toxic and non-immunogenic and results in long-lasting expression. The packaging capacity of recombinant AAV is 4.9 kb. Successful AAV-mediated gene transfer into brain, muscle, heart, liver, and lung tissue has been reported. Herpes simples type I (HSV) has a large genome (150 kb) and can transfer large intact genes. It has been used for gene transfer into neurons, tumors,

Available eukaryotic vectors include MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pET21b, pCEP4, and pEBVHis vectors. Most preferably, the MSCV virus can be used. Importantly, the vector should be such that the Omi family nucleic acid sequence is delivered to a cell and can be activated.

In this method, the eukaryotic vector contains an Omi nucleic acid sequence or variants thereof, wherein the Omi gene encodes an active or inactive Omi polypeptide molecule. The method involves isolating cDNA constructs from EST clones from full length Omi sequences, making Omi cDNA, PCR amplifying the Omi nucleic acid, and subcloning the Omi sequences in vectors, which are used to transfect host target cells. cDNA constructs are generated by obtaining an EST clone for a full-length human Omi WT or Omi family member. The EST is used as a DNA template for subcloning. An Omi family member cDNA is made, PCR amplified, and subcloned into selected sites of a vector. For example, the Nde I/Xho I sites of the pET21b vector can be used to generate C-terminal hexa-His tagged constructs.

An example related to an inactive Omi mutant construct starts with the construction of C-terminal c-Myc (SEQ ID NO. 80) tagged mammalian Omi expression vectors, the cDNA encoding the full-length Omi is PCR amplified. An Xba I-Kpn I fragment is inserted into a pcDNA 3.1 (−) vector through Xba I-Kpn I sites. The vector for the mature form of Omi (starting from AVPS) is generated similarly. An S306→Ala mutant is generated by replacing the BamH I/EcoR I fragment with a fragment containing the corresponding mutated codon. The mutation-containing fragment is obtained by BamH I/EcoR I digestion of the pET 21b vector for Omi S306→Ala. The fragment is then inserted into a pcDNA vector.

All of the above Omi nucleic acid sequence family members can be expressed by a recombinant cell, such as a bacterial cell, a cultured eukaryotic cell, or a cell of a non-human transgenic organism, such as a transgenic animal. Cultured cells available for use can include HEK 293 cells and U937 cells. Expression of Omi in a transgenic animal can be general or can be under the control of a tissue specific promoter. Preferably, one or more sequences, which encode an Omi polypeptide or a fragment thereof, are expressed in a preferred cell-type by a tissue specific promoter. Thus, once a vector is formed, any of a variety of cells can be transfected, including mammalian cells. A preferred cell is a mammalian, and more preferably, human tumor cell. In a preferred embodiment, the cell is a mammalian cell, especially a human cell. Exemplary cells include, for example, tumor cells, such as leukemic or carcinoma cells, or heart cells. For example, HEK cells can be transfected with any of a variety of the above vectors having an Omi family member.

The transfected cells include isolated in vitro populations of cells. In vivo, the vector can be delivered to selected cells, whereby the carrier for the vector is attracted to the selected cell population.

Activation of the gene in a transfected cell can be caused by an external stress factor. For example, the transfected cells can be contacted with an etoposide or a proteosome inhibitor. In the alternative, an activator can be included in the vector.

Probes or primers, which include or comprise a substantially purified Omi oligonucleotide can also be used herewith. The oligonucleotide includes a region of nucleotide sequence which hybridizes under stringent conditions to nucleotides of a selected Omi family member. Probes, or primers, can be used for a variety of applications, including for identification. In preferred embodiments, the purified nucleic acid is useful as a probe or primer; and has at least 50%, 60%, 70%, 80%, 90%, 95%, 98%, or 99% homology with a selected sequence of an Omi family member. The probe will be at least 10, 20, 30, 50, 100, or 200 nucleotides in length. In preferred embodiments, the probe or primer further includes a label. The label can be, for example, a radioisotope, a fluorescent compound, an enzyme, an enzyme co-factor, or combinations thereof.

A variety of polypeptides are expressed from the previously mentioned nucleic acid sequences. These Omi family polypeptides can be used to cleave IAP, to bind to LAP, to block binding to IAP, or a combination thereof. The polypeptide selected depends upon the desired use of the polypeptide. It is most preferred, however, to use the polypeptides to cleave IAP. The various polypeptides discussed below are known as Omi family polypeptides.

The Omi WT polypeptide has one or more of the following biological activities: 1) it interacts with, specifically binds to, a target, such as an apoptosis (caspase) inhibitor (IAP); 2) it proteolytically cleaves a substrate, such as IAP; 3) it is a serine protease; 4) it is a member of the MAP kinase cell signaling pathway; 5) it is involved in mammalian pathologies, such as ischemia of the kidney, the heart, or the forebrain; inflammatory response; septic shock; and, 6) it modulates a cellular response to stress. Most importantly, the Omi WT polypeptide binds to IAP and proteolytically cleaves IAP. The mature Omi WT polypeptide is SEQ ID NO. 44. The mature Omi WT polypeptide is comprised of 325 amino acids; however, some literature shows an Omi WT comprised of 458 amino acids. Like the nucleic acid sequences, the differences in the polypeptide number are trivial. The amino acid residues are cleaved prior to the AVPS region. Importantly, the protein or polypeptide has a catalytic triad, an AVPS region, a PDZ domain, and a hinge region. Omi is predominantly present in the intermembrane space of the mitochondria. It is released into the cytosol following apoptotic stimuli. The Omi WT polypeptide is derived from vertebrates, including primate and human organisms.

The Omi WT polypeptide can be cleaved or mutated to a variety of constructions. In particular, it can contain differing numbers of amino acid residues. An Omi WT polypeptide should, however, include the same protolytic activity as the Omi wild-type, SEQ ID NO. 44. This polypeptide includes an AVPS, hinge, PDZ, and catalytic triad domains. Variants that can be used will include different amino acids, deletions, or insertions, as compared to SEQ ID NO. 44. Variations of the Omi WT polypeptide, which maintain enzymatic activity in the catalytic triad, include SEQ ID NO. 45. SEQ ID NOs. 46 and 47 are inactive because the serine at position 173 has been substituted. The residues that are substituted are shown as part of the sequence listing.

An example of a formula, which illustrates Omi family polypeptides, is the formula C1ₙ₁-R1-C2ₙ₂-R2-C3ₙ₃-R3-4ₙ₄. In the formula, R1 is a serine; R2 is an amino acid residue selected from a group consisting of charged amino acid residues and aromatic amino acid residues; and, R3 is an amino acid residue selected from a group consisting of charged amino acid residues and polar amino acid residues. Residues R1, R2 and R3 form a catalytic triad for cleavage of the LAP. Specifically, R2 can be an amino acid residue selected from the group consisting of histidine, lysine, arginine, phenylalanine, tyrosine, and tryptophan. Specifically, R3 can be an amino acid residue selected from the group consisting of aspartic acid, glutamic acid, lysine, histidine, and arginine. The residue chains C1ₙ₁, C2ₙ₂, C3ₙ₃, and C4ₙ₄ are polypeptide chains, where the n1 subscript is equal to between 10 and 100, the n2 subscript is equal to between 10 and 100, the n3 subscript is equal to between 10 and 150, and the n4 subscript is equal to a number between 10 and 200. The C1ₙ₁, chain is the N-terminal and has an AVPS tetrapeptide motif sequence that operably couples to IAP. The C4ₙ₄ chain is the C-terminal and has a hinge sequence and PDZ domain. In preferred embodiments, the Omi WT polypeptide includes at least one IAP cleavage site. The R1 residue can be mutated from a serine to prevent IAP cleavage. Generally, the catalytic triad is formed from 3 residues at locations 65, 95, and 173.

Homologous and degenerate variants of the polypeptide discussed can be used. Suitable homologous sequences include those that maintain the proteolytic activity. Available homologous sequences include those that have proteolytic activity and at least 50% homology. It is more preferred to have at least 65% homology.

As mentioned, the Omi WT polypeptide can be used to cleave and degenerate IAP. It can be used as a basis for forming a small molecule for use in cleaving an IAP. A preferred small molecule will deliver the Omi polypeptide or variant to the target site. The Omi molecule can be used alone to cleave IAP or can be activated to cleave IAP. Use can occur in vitro or in vivo.

The PDZ domain regulates the enzymatic activity of the polypeptide. The PDZ region is approximately 100 amino acids in length. If the PDZ domain is removed, the polypeptide is more enzymatically active, meaning it more readily degrades IAP. As such, it may be preferred if the PDZ polypeptide can be removed or mutated. Additionally, substitutions can be made in the catalytic triad after removal or mutation of the PDZ polypeptide. When the PDZ domain is removed, the resultant polypeptide is known as an OmiΔPDZ polypeptide. In some cases, OmiΔPDZ polypeptide is preferred for use because of the increased proteolytic activity. SEQ ID NOs. 48-51 and 54-74 are variants of the OmiΔPDZ polypeptide. The OmiΔPDZ polypeptide without substitutions is SEQ ID NO. 48. SEQ ID NO. 76 is the PDZ peptide sequence. As mentioned, polypeptides can be produced whereby the triad has been substituted to produce an active or inactive polypeptide. Additionally, residues can be removed to shorten the polypeptide.

Amino acid substitutions of the wild-type Omi polypeptide can occur at positions 173 (serine), 95 (aspartic acid), and 65 (histidine) of SEQ ID NO. 44. If the serine is converted to a different amino acid, the expressed polypeptide will not enzymatically degrade IAP. Substitutions, however, can be made in any of the three residues, with the substitutions dependent upon the desired use of the polypeptide. The substitutions can inactivate the enzyme or can be such that enzymatic activity remains the same. The substitutions can be such that the amino acid residue has the same functionality, or a different functionality. As stated, these three amino acid residues form the catalytic triad. The substitutions are designed to allow for or eliminate protease activity. Members of the catalytictriad can be substituted, with the resultant protease activity remaining the same.

The Omi WT polypeptide can be manipulated, whereby the PDZ residues are eliminated. When the PDZ domain is eliminated, a more protealytically active polypeptide is formed. The PDZ domain regulates the enzymatic activity of the Omi polypeptide. SEQ ID NO. 48 has the PDZ domain eliminated from the Omi WT. In addition to removing the PDZ domain, the catalytic triad can be altered to have substitutions, as shown in SEQ ID NO. 49, where the polypeptide is active. When the PDZ domain is removed, it is known as an OmiΔPDZ polypeptide. In some cases, OmiΔPDZ polypeptide is preferred for use because of the increased proteolytic activity. SEQ ID NOs. 50 and 51 have the PDZ domain removed and express an inactive enzyme because of substitutions in the triad.

The Omi WT can be treated so that the AVPS peptide domain is eliminated or mutated. The AVPS tetrapeptide domain is the binding site for the LAP and competes with Smac for binding sites on the IAP.

When the AVPS domain is removed, the resultant molecule is known as OmiΔAVPS. The catalytic triad can be altered along with the removal or mutation of the AVPS nucleic acid sequence. SEQ ID NOs. 52 and 53 are the Omi polypeptide without AVPS. SEQ ID NO. 53 is the active Omi polypeptide with substitutions in the triad.

The Omi WT polypeptide can be mutated so that the AVPS and PDZ domains are eliminated. This is known as an Omi serine protease, wherein the sequence is without PDZ and AVPS domains. The hinge can also be removed. SEQ ID NO. 54 is the Omi serine protease polypeptide with AVPS and PDZ removed. Additionally, the catalytic triad can be altered or substituted concurrent with the removal of AVPS and PDZ. SEQ ID NO. 55 is an active Omi serine protease sequence with substitutions in the triad.

As mentioned, the hinge region can be removed from the Omi WT. The hinge region is associated with the PDZ domain, and helps regulate Omi. Typically, the hinge region is removed, or mutated with the PDZ region. SEQ ID NOs. 56-59 are polypeptides where the hinge and PDZ regions have been removed together. SEQ ID NO. 56 includes no substitutions to the triad and is active. SEQ ID NO. 57 includes substitutions in the triad and is active. SEQ ID NOs. 58 and 59 are inactive.

Finally, the Omi WT sequence can be of a short form, SEQ ID NO. 60 is an active sequence that does not include the hinge, PDZ, AVPS, and two residues, which form part of the residue chains. The triad in SEQ ID NO. 60 is not substituted; however, SEQ ID NO. 61 is short, active, and has substitutions in the triad. SEQ ID NOs. 62 and 63 are short, active enzymes. SEQ ID NOs. 64 and 65 are short, inactive enzymes.

SEQ ID NOs. 65-69 have the hinge and PDZ removed. Residues within the sequence are removed from the residue chains. The sequences are active, but have nucleotides, which encode residue chains deleted. SEQ ID NO. 70 has AVPS, the hinge, and PDZ removed along with two residues at the end. SEQ ID NOs. 71-75 are the same, but with triad additions or alterations.

The Omi WT can be treated so that the AVPS tetrapeptide (SEQ ID NO. 77) domain of the Omi polypeptide is eliminated. The AVPS domain is the binding site for the IAP and competes with Smac for the IAP binding site. The AVPS can be used alone to competitively bind with Smac or block Omi binding to IAP. SEQ ID NOs. 52 and 53 are the Omi polypeptide, without the AVPS tetrapeptide. Again, substitutions can be made, including to the catalytic triad, in addition to removing the AVPS peptides.

When the AVPS domain is removed, the resultant molecule is known as OmiΔAVPS. The catalytic triad can also be altered, along with the removal of the AVPS. SEQ ID NOs. 52-55 are Omi without the AVPS peptide sequence. The OmiΔAVPS region is 4 amino acid residues in length.

SEQ ID NOs. 54 and 55 are an Omi serine protease polypeptide. The protease region does not include the AVPS or PDZ domains. The Omi serine protease polypeptide is a vertebrate Omi serine protease polypeptide. Substitutions can be made to the triad within the serine protease polypeptide. The Omi serine protease region is approximately 221 amino acids in length. The region includes the catalytic triad. The triad folds into and cleaves target proteins.

The hinge polypeptide region can be removed from the Omi WT polypeptide. The hinge region is comprised of about 13 amino acid residues and is associated with the PDZ domain. Like PDZ, the hinge helps to regulate Omi. Typically, the hinge region is removed, or mutated with the PDZ region. Examples of sequences where hinge has been removed include SEQ ID NOs. 56-59. Like before, substitutions in the catalytic triad may occur.

The Omi catalytic triad polypeptide is similar to the protease domain and can have 208 amino acid residues as in SEQ ID NOs. 60 and 61. Importantly, the triad can have substitution or cleavage of the residue chains so that the “triad” includes the catalytic triad and at least one shortened chain of the amino acid chains attached to the triad. The Omi catalytic triad polypeptide is a vertebrate catalytic triad polypeptide.

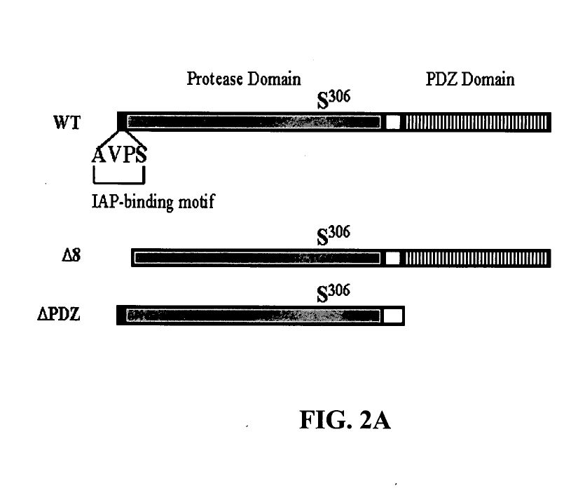

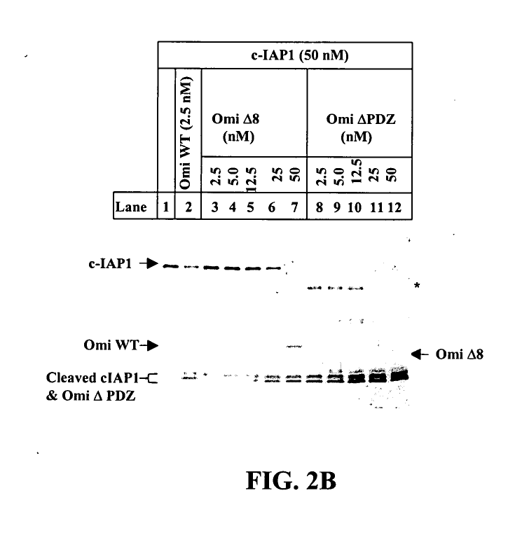

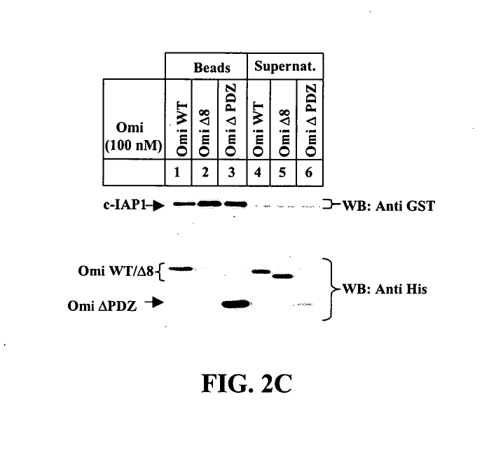

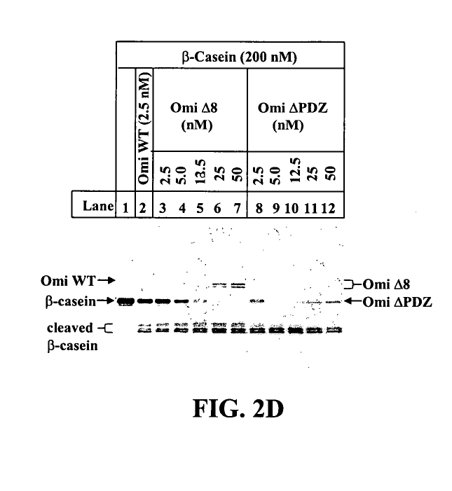

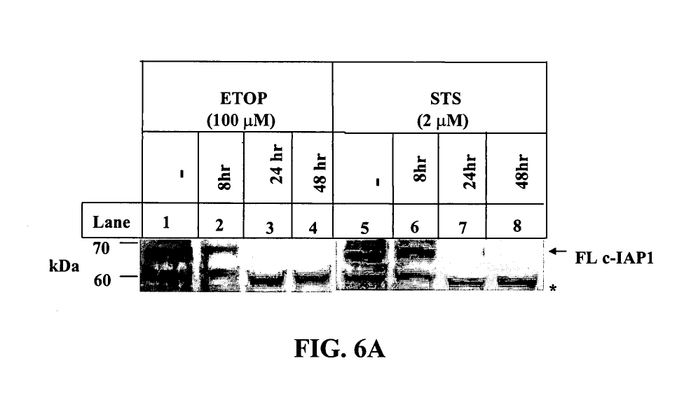

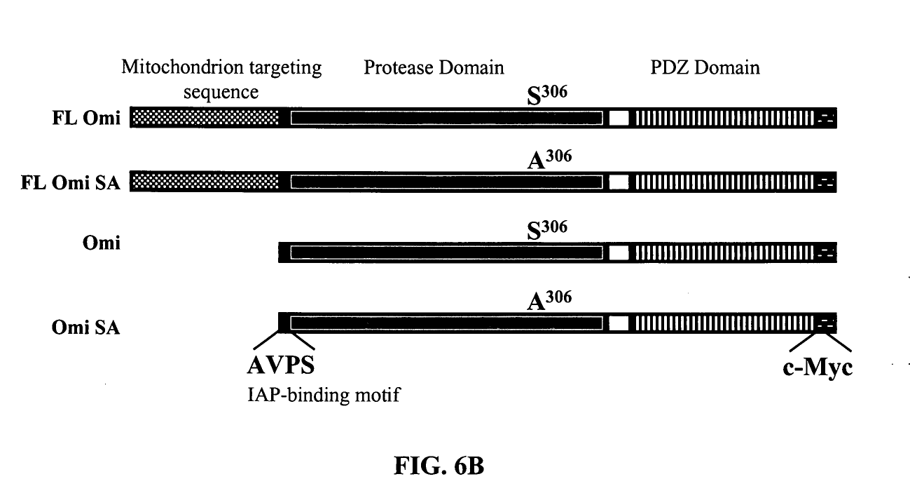

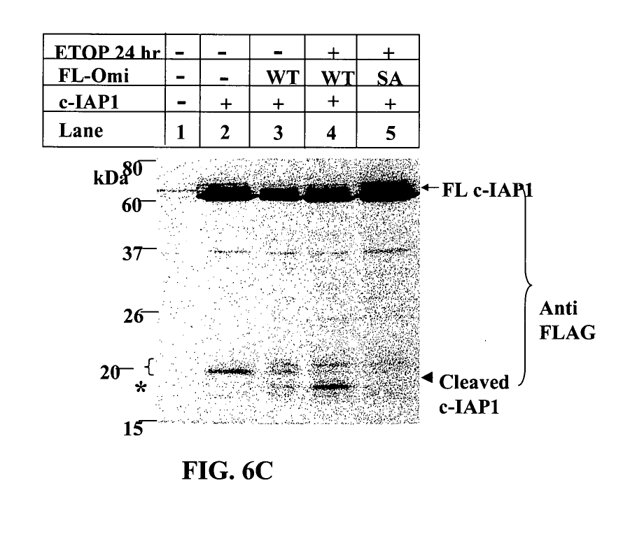

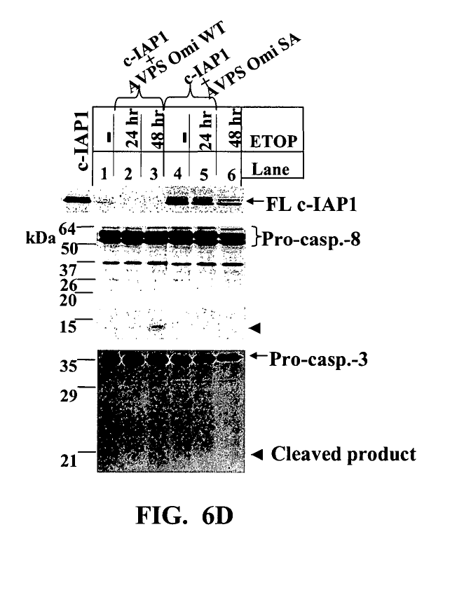

A schematic representation of Omi expression constructs where the upper two diagrams represent the full-length Omi constructs with the mitochondrial targeting sequences (MTS) at the N-terminal part of the molecule is shown in FIG. 6B. The lower two diagrams represent the cytosolic form of Omi with the IAP binding motif AVPS at the N-termini. The central region represents the protease domain with either a wild-type protease (S306) or an inactivated protease (A306). The hatched region represents the PDZ domain. The C-terminal end represents the engineered c-Myc tag in the constructs. The OmiΔ8 or ΔAVPS protein is also illustrated in FIG. 2A, with schematic representations of wild-type and the mutant form of Omi also shown in FIG. 2A. The N-terminal solid area represents the AVPS IAP binding motif. The serine protease domain is located in the central region of the molecule and the catalytic residue S306 is also shown. The C-terminal striped region represents the PDZ domain.

Homologous and substitution variants of the polypeptides can be used. Suitable homologous sequences include those that maintain the proteolytic activity. Available homologous sequences include those that have proteolytic activity and at least 50% homology. It is more preferred to have at least 65% homology.

Both WT and recombinant Omi polypeptides may be used, as long as the polypeptide cleaves IAP. The polypeptide can be obtained by isolation, or can be expressed by a recombinant cell. Alternatively, the polypeptide can be purchased. Mutant versions of the polypeptide can be obtained by forming a nucleic acid mutant and expressing the mutant.

Omi derived polypeptide fragments can be used where the fragment differs in amino acid sequence at up to 1, 2, 3, 5, or 10 residues, from the corresponding residues in SEQ ID NOs. 44-77. In other preferred embodiments, the fragment differs in amino acid sequence at up to 1%, 2%, 3%, 5%, 10%, 35%, or 50% of the residues from the corresponding residues in SEQ ID NOs. 44-77. In some embodiments, the differences are such that the fragment exhibits an Omi biological activity. In other embodiments, the differences are such that the fragment does not have Omi biological activity. In preferred embodiments, one or more, or all of the differences are conservative amino acid changes. In other embodiments, one or more, or all of the differences are other than conservative amino acid changes.

The Omi family polypeptide can include all or a fragment of an amino acid sequence from a selected sequence, fused, in a reading frame, to additional amino acid residues. As such, fusion proteins can be produced. In some embodiments, fusion molecules between the Omi serine protease catalytic triad and other biologically or enzymatically active serine proteases are made (e.g., human immunodeficiency virus serine protease, chymotrypsin, trypsin fusion molecules). In yet other preferred embodiments, the Omi polypeptide is a recombinant fusion protein having a first Omi portion and a second polypeptide portion, e.g., a second polypeptide portion having an amino acid sequence unrelated to Omi. The second polypeptide portion can be, e.g., any of glutathione-S-transferase, a DNA binding domain, or a polymerase activating domain. In a preferred embodiment, the fusion protein can be used in a two-hybrid assay. For example, a first Omi portion, e.g., an Omi portion containing a serine protease catalytic domain, e.g., amino acids 209 to end encoded by the last exon, can be fused to a DNA binding domain. In a two hybrid assay, the first Omi portion is co-expressed in a cell with a second polypeptide portion containing a transcription activation domain fused to an expression library, e.g., a HeLa cervical carcinoma expression library.

Polypeptides of the invention include those which arise as a result of the existence of multiple genes, alternative transcription events, alternative RNA splicing events, and alternative translational and postranslational events. The Omi polypeptide can be expressed in systems, for example cultured cells, which result in substantially the same postranslational modifications present when expressed Omi is expressed in a native cell, or in systems which result in the omission of postranslational modifications present when expressed in a native cell.

The invention includes an immunogen, which includes an Omi polypeptide in an immunogenic preparation, the immunogen being capable of eliciting an immune response specific for the Omi polypeptide. For example, a humoral immune response, an antibody response, or a cellular immune response can be elicited. In preferred embodiments, the immunogen comprises an antigenic determinant, such as a unique determinant, from a protein represented by SEQ ID NO. 44.

The present invention also includes an antibody preparation specifically reactive with an epitope of the Omi immunogen or generally of an Omi polypeptide, preferably an epitope which consists all or in part of residues from the amino acid sequence SEQ ID NO. 44, or an epitope, which when bound to an antibody, results in the modulation of a biological activity.

Thus, the Omi WT or recombinase polypeptide, as expressed in the cells in which it is normally expressed or in other eukaryotic cells, has a molecular weight of about 57 kDa, as estimated from the nucleic acid sequence SEQ ID NO. 1. The recombinant Omi polypeptide has one or more of the following characteristics:
(i) it is approximately 529 amino acids in length;
(ii) it has the ability to cleave a substrate, e.g., a protein;
(iii) it has a molecular weight, amino acid composition or other physical characteristic of SEQ ID NO. 44;
(iv) it has an overall sequence similarity of at least 50%, preferably at least 60%, more preferably at least 70%, 80%, 90%, or 95%, with SEQ ID NO. 44;
(v) it is found in all human tissues;
(vi) it has at least one PDZ domain, which is preferably about 70%, 80%, 90%, or 95% identical to SEQ ID NO. 76;
(vii) it has an AVPS domain, which is preferably about 70%, 80%, 90%, or 95% identical to SEQ ID NO. 77; and,
(viii) it has a carboxy terminal serine protease catalytic domain containing at least one site of serine protease activity, which is preferably about 70%, 80%, 90%, or 95% identical to amino acid residues 181-529 of SEQ ID NO. 60.

Also included in the invention is a composition which includes either a nucleic acid sequence encoding the Omi family molecule or variants thereof, or an Omi-derived polypeptide, together with one or more additional components, such as a carrier, diluent, or solvent. The additional component can be one which renders the composition useful for in vitro and in vivo pharmaceutical or veterinary use.

The Omi nucleotide sequence can be delivered by mechanical, electrical or chemical procedures to target cells. Mechanical methods include microinjection, pressure, and particle bombardment. Electrical methods include electroporation. Chemical methods for Omi nucleotide delivery can utilize liposomes, DEAE-dextran, calcium phosphate, artificial lipids, proteins, dendrimers, or other polymers, including controlled-release polymers. Direct microinjection of an Omi family member nucleotide can be utilized in vitro.

Mechanical methods can be utilized, such as hydrodynamic force and other external pressure-mediated DNA transfection methods. Alternatively, ultrasonic nebulization can be utilized for DNA-lipid complex delivery. Particle bombardment, also known as biolistical particle delivery, can introduce DNA into several cells simultaneously. Widely used in DNA vaccination procedures, DNA-coated microparticles (e.g., gold, tungsten) are accelerated to high velocity to penetrate cell membranes or cell walls. This procedure is used predominantly in vitro for adherent cell culture transfection.

Electroporation, using high-voltage electrical impulses to transiently permeabilize cell membranes, permits cellular uptake of macromolecules, such as nucleic acid and polypeptide sequences. Thus, Omi nucleic acid molecules can be inserted into cells by electroporation. In this method, Omi nucleic acid sequences would be inserted into target cells in vitro or in vivo using voltage current.

Chemical methods, using uptake-enhancing chemicals are effective drug delivery systems. For nucleotides, positively charged chemicals, usually polymers, interact with negatively charged nucleotide molecules. DEAE-dextran and calcium phosphate, interacting to form DEAE-dextran-DNA and calcium phosphate-DNA complexes respectively, permit deposition of complexes onto cell surfaces, and internalization into the cell by endocytosis.

Lipofectin-DNA is an artificial lipid-based DNA delivery system. Liposomes (either cationic, anionic, or neutral) are complexed with DNA. The liposomes can be used to enclose an Omi nucleic acid for delivery to target cells, in part, because of increased transfection efficiency.

Protein-based methods for DNA delivery, perhaps with addition of other chemicals, are also utilized. The cationic peptide poly-L-lysine (PLL) can condense DNA for more efficient uptake by cells. PLL has been conjugated with ligands, such as asilo-orosomucoid (ASOR), which binds to a liver-specific asialo-glycoprotein to achieve receptor-mediated uptake. Protamine sulfate, polyamidoamine dendrimers, and synthetic polymers, and pyridinium surfactants have also been utilized.

Biocompatible controlled-release polymers have recently been examined. Biodegradable poly (D,L-lactide-co-glycolide) microparticles and PLGA microspheres have been used for long-term controlled release of DNA molecules to cells. DNA has also been encapsulated into poly(ethylene-co-vinyl acetate) matrices, resulting in long term controlled, predictable release for several months. Omi DNA-derived particles can be made for controlled-release.

A suitable drug delivery system will possess the following properties: ease of packaging assembly of Omi nucleic acid; delivery to target cells leading to high transfection efficiencies; stabilization of DNA molecules, bypassing or escaping from cellular endocytotic degradative pathways; efficient decomplexation or unpackaging of DNA upon intracellular release; efficient nuclear targeting of Omi DNA; and high, persistent, and controllable expression of therapeutic levels of Omi proteins.

Similarly, the Omi polypeptide, or variants thereof, can also be delivered to target cells by mechanical, electrical or chemical means. Mechanical methods include microinjection, pressure, and particle bombardment. Direct microinjection of Omi polypeptide into cells in vitro occurs directly and efficiently. As with DNA-injected cells, once cells are modified in vitro, they can be transferred to the in vivo host environment. In particle bombardment, Omi polypeptide-coated microparticles are physically hurled with force against cell membranes or cell walls to penetrate cells in vitro. Hydrodynamic force, pressure-mediated methods, and ultrasonic nebulization may also be used to permit Omi polypeptide penetration into cells.

Electroporation, particularly with low voltage, high frequency electrical impulses, can be used for Omi polypeptide insertion into cells both in vivo and in vitro. Chemical methods are equally utilizable for Omi polypeptide molecules. Liposomes are a preferred method for enclosure of Omi polypeptides for delivery to target cells. These liposomes may be armed with target-specific antibodies or other targeting molecules (e.g., asilo-orosomucoid, ASOR). Biocompatible controlled-release polymers, such as biodegradable poly (D,L-lactide-co-glycolide) microparticles and PLGA microspheres, or alternative microspheric structures can be used with Omi polypeptide. These Omi polypeptide-containing particles may be used for in vivo treatment protocols. Cationic peptides (e.g. PLL) might also be used with predominantly anionically charged Omi polypeptides.

A preferred synthetic Omi polypeptide drug delivery system should possess the following features: ease of packaging of Omi polypeptide; delivery to target cells with high efficiency; readily transferred across membranes to exist intracellularly; efficient decomplexation or “unpackaging” of Omi upon cytosolic release; efficient targeting to LAP; and high, persistent, and controlled targeting of therapeutic levels of Omi polypeptide to target cells.

Since the Omi polypeptide has been characterized as triggering apoptosis or cell death, it can be used in in vivo and in vitro treatment of tumors. For example, anti-idiotype Fab-bearing Omi-containing liposomes can be used to target idiotype-bearing human B leukemia cells. In in vitro studies, chromium-51 release from targeted leukemia cells can be measured. In in vivo studies, reduction in the number of anti-idiotype-bearing B leukemia cells, upon treatment with armed Omi-containing liposomes, can be measured by fluorescent activated cell sorter (FACS) methods using anti-idiotype antibody to detect the B cell idiotypic marker or tumor cells.

cIP1, cIAP2, XIAP, Livin α, and Livin β are identified as the serine protease substrates of Omi/HtrA2. Omi/HtrA2 catalytically hydrolyzes these IAPs by its catalytic residue S306, and the catalytic activity for IAPs is completely diminished in the active site mutant. The Omi/HtrA2-catalyzed IAP cleavage has been shown to be 10-fold enhanced by the specific binding between Omi/HtrA2 and IAPs mediated by the AVPS motif on Omi/HtrA2. Omi/HtrA2 has a novel function for catalytically hydrolyzing IAPs and, thus, lowering caspase inhibition, as well as an ability to degrade caspases. The final effect is to promote cell death.

The IAP molecule is found primarily in eukaryotic cells. The LAP is derived from cells selected from the group consisting of mammalian, reptile, aves, and amphibian cells. IAP molecules cleaved by Omi include cIP1, cIAP2, XIAP, Livin α, Livin β, and DIAP1. The IAP that is cleaved by the Omi polypeptide is BIR2 deficient.

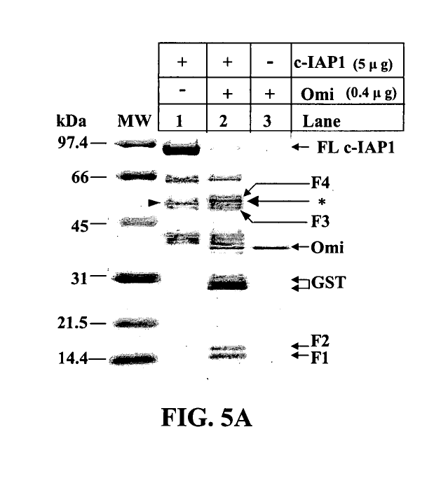

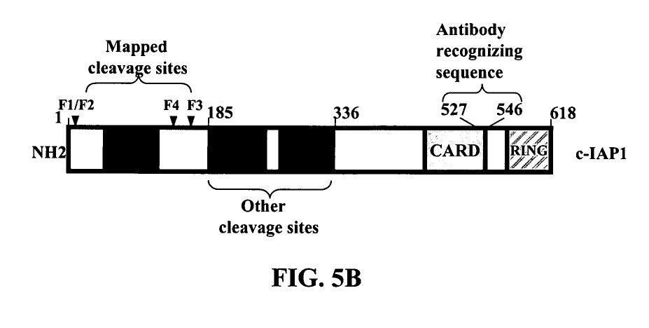

In view of the above, a method for cleaving IAP in vitro can be practiced, whereby IAP is cleaved and causes caspase activation when IAP is bound to a caspase. The method is initiated by contacting an amount of IAP bound to a caspase with an amount of an active Omi family polypeptide. Upon contact, Omi will cleave IAP and release the caspase from IAP. The cleavage sites of IAP include those shown in FIG. 5B, whereby any Omi polypeptide that cleaves one of the selected sites is suitable for use. The Omi to IAP molar ratio in vitro is equal to between 1:5 to 1:30 molar ratio of Omi to IAP. The in vitro conditions include an incubation time of 2 hours at 37° C. in solution. As such, contact is sufficient to cause cleavage. Further, IAP is more readily cleaved by OmiΔPDZ.

In vivo cleavage of IAP can be accomplished by transfecting a mammalian cell with an Omi vector. The Omi vector will include an active Omi family nucleic acid sequence. As such, the sequence will express a polypeptide that will cleave IAP. Once a population of cells has been transfected with a population of Omi vectors, Omi expression can be stimulated by treating the cells with an etoposide, or similar composition, which causes a stress response. Expression can be caused by the addition of etoposide or damage to the cell. As such, Omi will be expressed and will cleave IAP. An alternative way to cleave IAP in vivo is to use a carrier, such as a liposome, with an Omi polypeptide. The carrier, or liposome, will transport the Omi across the cell membrane and place Omi in contact with LAP. Again, upon contact, cleavage of IAP will occur. Obviously, other carriers, other than liposomes, can be used to transport active Omi polypeptide into a desired cell.

An alternative method involves contacting cells having LAP with a recombinant cell that expresses Omi. The Omi family polypeptide can be expressed by a nucleic acid sequence molecule previously mentioned.

The Omi polypeptide can be used as part of a method for preventing IAP ubiquitination of caspase. This will result in caspase activation. Thus, a method for promoting apoptosis and causing caspase activation can be practiced. As stated, IAP is bound to a caspase and is contacted with an amount of an Omi polypeptide, whereby upon contact, Omi will cleave IAP and release the caspase from IAP.

A hybridization kit can be made for detecting an Omi wild-type gene, wherein the kit comprises a container and an Omi nucleic acid molecule including Omi family nucleic acid sequences. Preferably, the kit has a container and a nucleic acid molecule, which includes one of the nucleotide molecules of SEQ ID NOs. 1-41. A kit for detecting an Omi gene comprising PCR primers spanning an Omi gene or related Omi gene can be made. The kit will include a positive control, and sequencing products.

The following definitions define terms used herein:

Allele is a shorthand form for allelomorph, which is one of a series of possible alternative forms for a given gene differing in the DNA sequence and affecting the functioning of a single product.

An amino acid (aminocarboxylic acid) is a component of proteins and peptides. Joining together of amino acids forms polypeptides. Polymers containing 50 or more amino acids are called proteins. All amino acids contain a central carbon atom to which an amino group, a carboxyl group, and a hydrogen atom are attached. Protein molecules can be referred to as polypeptides when the protein molecule is less than 500 amino acids in length.

An antigen (Ag) is any molecule that can bind specifically to an antibody (Ab). Their name arises from their ability to generate antibodies. Each Ab molecule has a unique Ag binding pocket that enables it to bind specifically to its corresponding antigen. Abs are produced by B cells and plasma cells in response to infection or immunization, bind to and neutralize pathogens, or prepare them for uptake and destruction by phagocytes.

Caspase is defined as a group of cysteine proteases involved in apoptosis.

Chimera is an individual composed of a mixture of genetically different cells. The genetically different cells of chimeras are required to be derived from genetically different zygotes.

DNA cassette is a deoxyribonucleic acid (DNA) sequence that can be inserted into a cell's DNA sequence. The cell in which the DNA cassette is inserted can be a prokaryotic or eukaryotic cell. The prokaryotic cell may be a bacterial cell. The DNA cassette may include one or more markers, such as Neo and/or LacZ. The cassette may contain stop codons. In particular, a Neo-LacZ cassette is a DNA cassette that can be inserted into a cell's DNA sequence located in a bacterial artificial chromosome (BAC). Such DNA cassettes can be used in homologous recombination to insert specific DNA sequences into targeted areas in known genes.

Degenerate code is one in which each different word is coded by a variety of symbols or groups of letters. The genetic code is said to be degenerate because more than one nucleotide triplet codes for the same amino acid.

A gene is a hereditary unit that has one or more specific effects upon the phenotype of the organism that can mutate to various allelic forms.

Homologous chromosomes are chromosomes that pair during meiosis. Each homolog is a duplicate of one of the chromosomes contributed at syngamy by the mother or father. Homologous chromosomes contain the same linear sequence of genes and, as a consequence, each gene is present in duplicate.

A host organism is an organism that receives a foreign biological molecule, including an antibody or genetic construct, such as a vector containing a gene.

Mutation is defined as a phenotypic variant resulting from a changed or new gene.

Mutant is an organism bearing a mutant gene that expresses itself in the phenotype of the organism. Mutants include both changes to a nucleic acid sequence, as well as elimination of a sequence or a part of a sequence. In addition polypeptides can be expressed from the mutants.

A nucleic acid is a nucleotide polymer better known as one of the monomeric units from which DNA or RNA polymers are constructed, it consists of a purine or pyrimidine base, a pentose, and a phosphoric acid group.

Omi/HtrA2 is a polypeptide which causes activation of caspase.

Peptide is defined as a compound formed of two or more amino acids, with an amino acid defined according to standard definitions, such as is found in the book “A Dictionary of Genetics” by King and Stansfield.

Plasmids are double-stranded, closed DNA molecules ranging in size from 1 to 200 kilobases. Plasmids are incorporated into vectors for transfecting a host with a nucleic acid molecule.

A polypeptide is a polymer made up of less than 350 amino acids.

Protein is defined as a molecule composed of one or more polypeptide chains, each composed of a linear chain of amino acids covalently linked by peptide bonds. Most proteins have a mass between 10 and 100 kilodaltons. A protein is often symbolized by its mass in kDa.

Smac stands for the second mitochondria-derived activator of caspase, after cytochrome c.

Small molecules are defined as regulatory polypeptide or nucleic acid molecules that cause detectable changes in protein-protein interaction systems that may also affect one or more phenotypic changes. These small molecules may operatively function by structural similarity to and competitive inhibition with native molecules in vitro or in vivo. Phenotypic changes may include observed changes in such parameters as HSC-proliferation, bone deposition or bone mineral density, tooth development, and ocular development. Small regulatory polypeptide molecules include, but are not limited to, antibody fragments such as Fab, F(ab)₂, Fv, and antibody combining regions. Small regulatory nucleic acid molecules include, but are not limited to, antisense RNA sequences that interfere with wild-type polypeptide function; and truncated nucleic acid sequences that encode shortened polypeptides that interfere with function.

Support is defined as establishing viability, growth, proliferation, self-renewal, maturation, differentiation, and combinations thereof, in a cell. In particular, to support an HSC population refers to promoting viability, growth, proliferation, self-renewal, maturation, differentiation, and combinations thereof, in the HSC population. Support of a cell may occur in vivo or in vitro.

A vector is a self-replication DNA molecule that transfers a DNA segment to a host cell.

Wild-type is the most frequently observed phenotype, or the one arbitrarily designated as “normal”. Often symbolized by “+” or “WT.”

### EXAMPLES

### Example 1

The present example relates to the identification of substrates targeted by the serine protease, Omi/HtrA2. In particular, the present example relates to the serine protease activity of Omi. An analysis was directed to whether IAP is an enzymatic target of Omi. This possibility was tested in an Omi-catalyzed serine protease reaction in vitro using purified recombinant proteins. As will be shown, the mutant form of Omi that is deficient in IAP binding still bears the protease function and can induce cell death through a caspase-mediated pathway.

Since Omi promotes cell death through its serine protease activity, it was examined to determine if its serine protease activity was responsible for hydrolyzing IAPs. It is known that the enzymatic active site of Omi resides at S306, and the mutation of S306 to Alanine completely abolishes Omi's serine protease activity for the generic substrate β-casein.

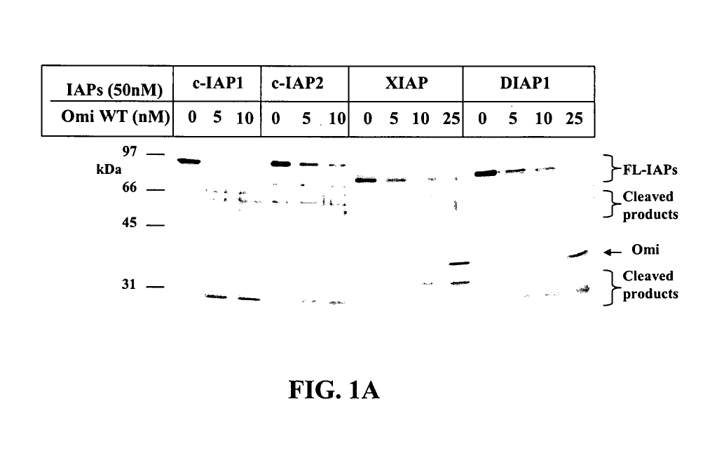

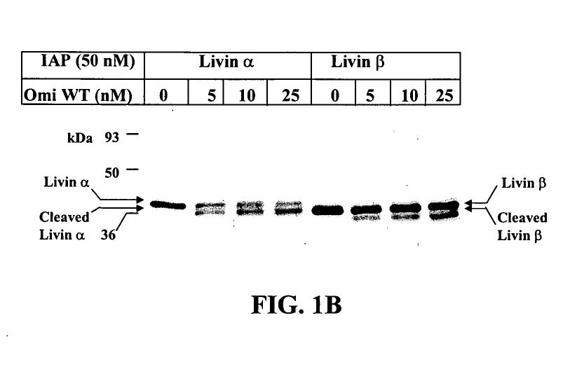

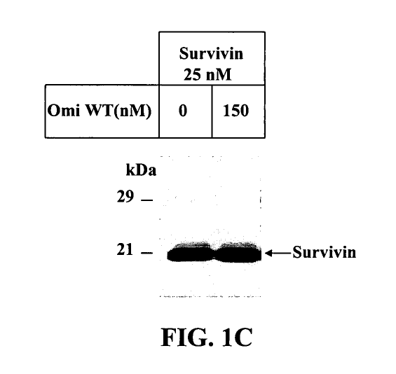

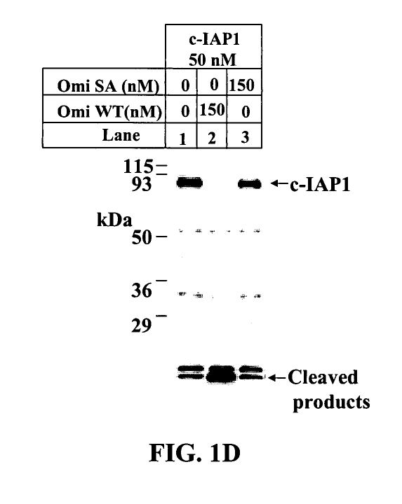

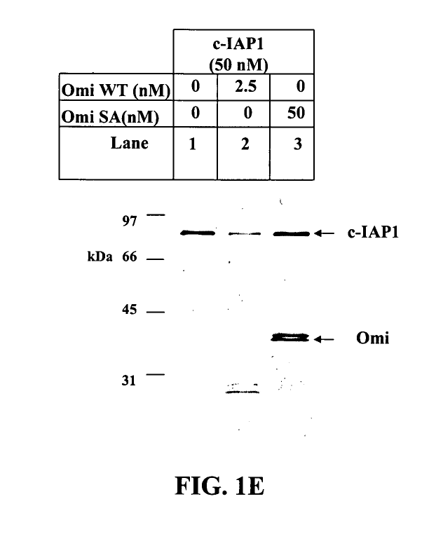

Different IAP proteins were incubated with wild-type (WT) Omi and the serine protease mutant of Omi (Omi SA) for 2 hours at 37° C. in 40 μl of PBST. The various IAPs included cIAP1, cIAP2, XIAP and DIAP1 (50 nM each), and were incubated with increasing amounts of Omi WT (0-150 nM). The reaction mixtures were resolved by 13.5% SDS-PAGE and followed by a Western blot with anti-GST antibody to detect the integrity of the GST-fused cIAP1 molecule. The HRP-conjugated antibody against GST was used since cIAP1 was a GST fusion protein. As shown in FIG. 1A, Omi WT cleaved IAP proteins.

Next, Livin α and Livin β (50 nM each) were incubated with increasing amounts of Omi WT (0-25 nM), as shown in FIG. 1B. The samples were resolved by 13.5% SDS-PAGE. The cleavage products were detected by Western blotting with an antibody against Livin, since Omi co-migrated with Livin in the gel and this could interfere with the identification of Livin cleavage by silver staining. As can be seen, Omi WT cleaved Livin α and Livin β.

Survivin (25 nM) was incubated with excessive amounts of Omi WT (150 nM), as shown in FIG. 1C. The samples were separated on 13.5% gel and immunoblotted with an antibody against Survivin.

cIAP1 (50 nM) was incubated with 150 nM of Omi WT (Lane 2) and Omi SA (Lane 3), as shown in FIG. 1D. Omi WT cleaved the cIAP1, but the Omi SA did not. As shown in FIGS. 1A-D, the wild-type Omi cleaved various forms of IAP proteins, including cIAP1, cIAP2, XIAP, Livin α and Livin β. This cleavage activity, however, was absent for Survivin. The Omi mutant (OmiSA), as seen in FIG. 1D, did not cleave the IAP molecule. Therefore, it was concluded that the IAP cleavage activity by Omi is dependent on its serine protease activity. Taken together, cIAP1, cIAP2, XIAP, Livin α and Livin β are a group of proteolytic substrates of Omi in vitro, and this proteolysis is conferred by the serine protease of Omi.

### Example 2

The tetrapeptide AVPS at the N-terminus of processed Omi serves as the IAP binding motif. The AVPS tetrapeptide is shown in FIG. 2A. IAP binding is a prerequisite for Omi to release the IAP-bound caspases and cause reactivation of the caspases. In order to examine if IAP binding is also required for Omi to catalytically hydrolyze its IAP substrates, an Omi variant that was unable to bind to IAPs was tested. The Omi variant retained its serine protease activity. The Omi molecule without the AVPS tetrapeptide is known as OmiΔ8.

50 nM of cIAP and 200 nM of β-casein were incubated with 2.5 nM of wild-type Omi (FIG. 2B, lane 2) and varying amounts of OmiΔ8 mutant (FIG. 2B, lanes 4-8) in a final volume of 50 μl PBST. After incubation for 2 hours at 30° C., one third of each sample was subjected to SDS-PAGE followed by silver staining. As shown in FIG. 2B, OmiΔ8 did not cleave as efficiently as Omi WT, regardless of the substrate targeted.

Wild-type Omi (Omi WT) protein at 2.5 nM (nano Molar) almost completely cleaved 50 nM cIAP1, which was at a 1:20 molar ratio of enzyme versus substrate (FIG. 2B, lane 2). In contrast to the Omi WT, the OmiΔ8 protein at the same concentration could not completely cleave cIAP1. A concentration (25 nM), 10-fold higher than the wild-type, was required to cleave IAP, which was at a 1:2 molar ratio of enzyme versus substrate (FIG. 2B, lane 6 and 7). The proteolytic efficiency of OmiΔ8 was, therefore, 10-fold lower than that of Omi WT regarding cIAP1 cleavage.

To examine whether the differential in catalytic efficiency between Omi WT and OmiΔ8 was due to different binding affinities to IAPs, a GST-based pulldown assay was used to monitor the IAP binding affinities of Omi WT and OmiΔ8. Full-length cIAP1 (50 nM) was incubated with 100 nM of Omi WT and Omi mutants for 20 minutes at 4° C. in 50 μl of PBST. The samples were then incubated with 20 μl of Glutathione Sepharose beads (Amersham Biosciences, Piscataway, N.J.) for 30 minutes at 4° C. The beads were precipitated by centrifugation and washed briefly with 1.4 ml×3 of PBST. The proteins bound to the beads and left in the supernatant were separately mixed with SDS sample loading buffer and resolved by SDS-PAGE, then transferred to a nitrocellulose filter. The upper part of the filter was probed with an antibody against GST and the lower part with an antibody against Penta-His. As shown in FIG. 2C, Omi WT could bind to a cIAP1, whereas OmiΔ8 completely lost IAP binding, which correlated with the catalytic activity. This indicated that the direct binding of Omi to IAPs was required for Omi to efficiently cleave IAPs, and this IAP binding motif-directed association between Omi and cIAP1 greatly accelerated the proteolytic efficiency for cIAP1.

To further investigate whether the AVPS-mediated binding of Omi to its substrates is a common mechanism for Omi's protease functionality, cleaving of β-casein by Omi WT and OmiΔ8 was assayed. In contrast to cIAP1 cleavage, the same amount of β-casein was cleaved by wild-type Omi WT and OmiΔ8 at 2.5 nM, as shown at FIG. 2D, lanes 2 and 4. The cleavage efficiciency for β-casein, therefore, showed no difference between wild-type and the AVPS-deficient mutant of Omi (OmiΔ8). This indicates that the proteolytic activity of Omi on β-casein cleavage is not dependent on the AVPS-mediated binding. It can be concluded that, in contrast to this apoptosis irrelevant substrate, Omi utilizes the AVPS-mediated binding as a regulatory mechanism to selectively bind to IAPs which, in turn, substantially enhances the catalytic efficiency of Omi (at least 10-fold) for IAP proteolysis.

### Example 3

Other than the N-terminal AVPS motif for IAP binding and the central protease domain, Omi also carries one other domain that is important for its function. The other region is the PDZ domain at the C-terminal region of the molecule. Studies from the Omi crystal structure have illustrated that the molecular composition of native Omi protein is a homotrimer that is constituted mainly through the binding among its protease domains. The PDZ domain of Omi temporally restricts the substrate accessibility to the active site of the Omi serine protease domain. Deletion of the PDZ domain consequently results in a higher protease activity in β-casein cleavage.

To examine the effect of the PDZ domain of Omi on cIAP1 cleavage, a mutant form of Omi, whereby the PDZ domain (OmiΔPDZ) was deleted, was used to compare its cleavage efficiency versus Omi WT. The conditions were the same in vitro conditions listed in Example 1. The OmiΔPDZ at 2.5 nM cleaved the full-length cIAP1 molecules into smaller fragments, whereas the Omi WT enzyme, at the same concentration, still left some uncleaved full-length cIAP1 molecules, shown at FIG. 2B, lane 2 versus lane 8. The higher proteolytic efficiency of OmiΔPDZ was contributed, at least in part, by its higher binding affinity to cIAP1, as shown in a GST-pulldown assay (compare lane 3 with lane 1 in panel C).

The cleavage activity of β-casein by OmiΔPDZ was also stronger than by Omi WT, as shown in FIG. 2D, indicating that no matter whether the substrate is related to apoptosis or not, OmiΔPDZ possessed a higher proteolytic activity than the wild-type enzyme for both cIAP1 and β-casein. This indicates that the PDZ domain of Omi is probably not involved in substrate recognition, but functions to modulate the scale of the serine protease activity by regulating either the accessibility of substrates to the serine protease domain, the catalytic activity of the protease, or both. The critical regulation is at the recognition step between the Omi enzyme and the substrates, which is through the IAP binding motif-mediated association between Omi and IAPs.

### Example 4

Since Omi and Smac bind to IAP proteins through the N-terminal conserved tetrapeptide AVPS and AVPI, respectively, it is reasonable to speculate that there are some functional interactions between Omi and Smac in the context of IAP cleavage. This was analyzed first by determining if Omi could proteolytically process Smac. Not surprisingly, when incubated with Omi, either in the presence or absence or cIAP1, Smac stayed in its unprocessed form throughout the reaction. In an immunoprecipitation assay, Smac and Omi did not bind to each other regardless of the presence of cIAP1. This result excluded the possibility of a direct interaction between the two molecules. Smac, therefore, was not cleavable by Omi. The SDS-PAGE and Western Blot data is not shown.

### Example 5

The IAP family of proteins regulate caspase activity by two mechanisms. They can either directly inhibit active caspases via their BIR domains or degrade caspases via their RING domain-mediated caspase ubiquitination. The caspase inhibitory activity and ubiquitin ligase activity of cIAP1 was compared before and after cleavage by Omi.

cIAP1 protein (400 nM) was incubated with varying amounts of Smac, Omi WT or Omi SA for 2 hours at 37° C. in 10 μl of buffer. This incubation was to generate Omi-cleaved cIAP1 with Smac and Omi SA serving as negative cleavage controls. These samples were subsequently tested for their caspase reactivation activity by incubating with an equal volume of HeLa S1100 extracts supplemented with 1 mM MgCl₂, 1 mM DATP, 24 ng/μl cytochrome c, 1 mM DTT (final concentration) and a proper amount of ³⁵S-procaspase-3 for 40 minutes at 30° C. The reactions were stopped by adding 7 μl of 4×SDS sample buffer. The proteins were subjected to 13.5% SDS-PAGE and transferred to the nitrocellulose filter. The filter was first exposed to a phosphor screen for one hour at room temperature (upper panel) to reveal the caspase activity; then the filter was probed with HRP conjugated antibody against GST to check the cleavage of cIAP1 (lower panel).

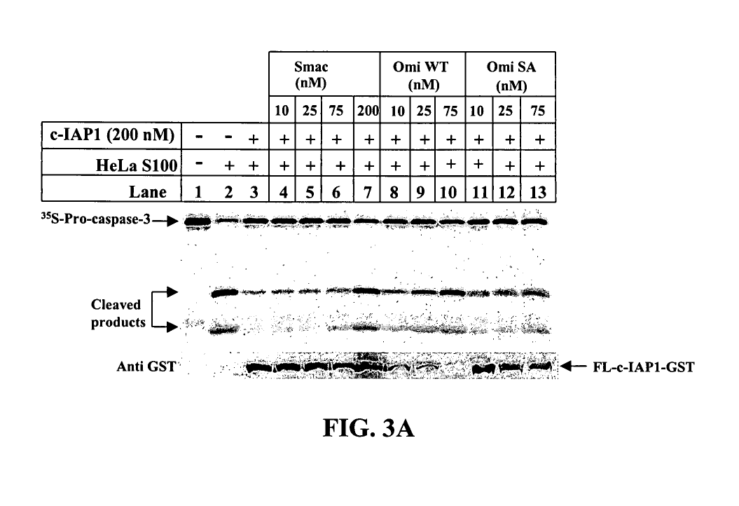

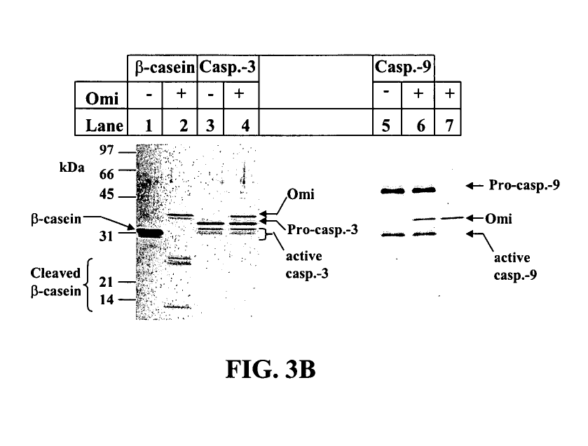

Recombinant Omi proteins were tested to determine whether they were able to release IAP-inhibited caspases using Smac as the positive control. The addition of dATP and cytochrome c to HeLa cell extracts triggered the activation of endogenous caspase-9, which can be measured by the cleavage of S-labeled procaspase-3, illustrated at FIG. 3A, lane 2. Caspase activity was completely inhibited by 250 nM of cIAP1, shown in FIG. 3A, lane 3. The IAP inhibition was relieved by 200 nM of Smac, shown in FIG. 3A, lane 7. cIAP1 inhibition was reduced by Omi at 10 nM and relieved at 75 nM, whereas the protease dead mutant Omi (Omi SA) just started to reduce the inhibition at 75 nM, as shown in FIG. 3A, lanes 8-13. The cIAP1 cleavage was further confirmed by Western blotting (lower part, panel A). Like Smac, Omi WT was able to reactivate cIAP1-inhibited caspase-9 (lanes 5, 6, and 8).

About 250 ng of either recombinant caspase-3 (lane 4) or caspase-9 (lane 6) was incubated with 50 ng of Omi at 37° C. for 2 hours in a final volume of 40 μl PBST. Omi cleavage of β-casein was included as a positive control (lane 2). The reaction mixtures were resolved by electrophoresis on a 7.5-20% gradient gel and visualized by silver staining. All of the samples were run on the same gel. The splitting of the gel into two parts in this figure presentation was for the convenience of sample labeling. The two parts, therefore, shared the molecular weight marker. Caspase-9 and caspase-3, in either the pro-form or the active form, were not cleaved by Omi, as shown in FIG. 3B. Thus, the caspase activity generated by Omi was due to Omi cleavage of cIAP1. Therefore, Smac stoichiometrically antagonizes cIAP1 exclusively through direct binding of its N-terminus to IAPs. The binding-directed Omi cleavage of IAPs, on the other hand, is catalytic and irreversible, thereby more efficiently inactivating IAPs.

The anti-caspase activity of the cleaved cIAP1 was dramatically attenuated, as shown in FIG. 3. In FIG. 3, boxes are included which have columns and rows, which relate to the various enzymatic substrates and the forms of Omi. A (−) indicates that the particular form of Omi was not present. A (+) indicates its presence.

### Example 6

The ubiquitin ligase activity of cleaved cIAP1 on caspase substrates was analyzed. It was concluded that cIAP1 cleavage by Omi/HtrA2 attenuates the cIAP1 Ub ligase activity on caspase substrates.

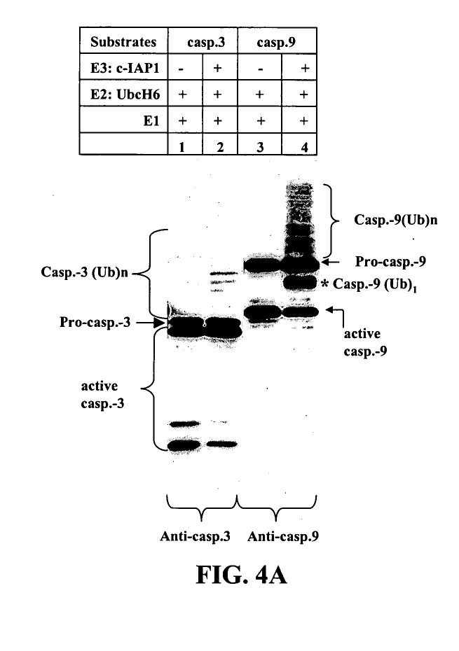

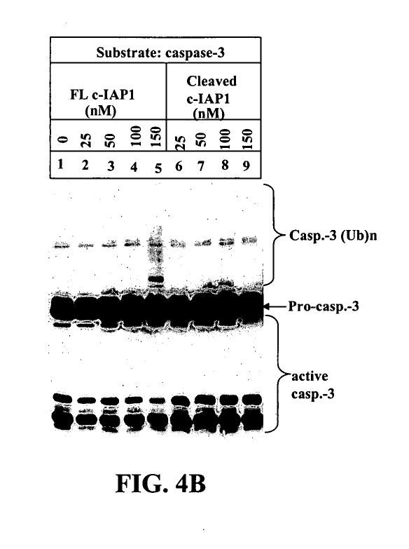

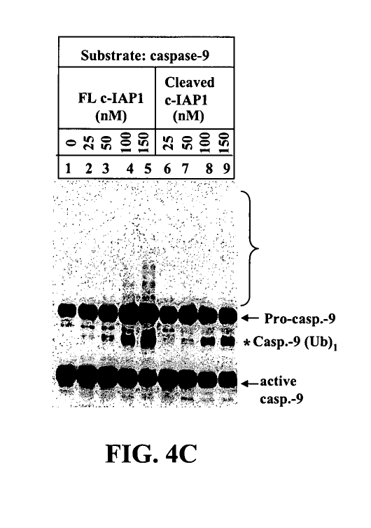

The ubiquitin ligase activity of cIAP1 was measured with a total reconstituted in vitro system, consisting of purified protein factors required for ubiquitin conjugation reaction. When ubiquitin and caspase-3 or caspase-9 were incubated with a yeast E1 enzyme, an E2 (UbCH6) enzyme, and an E3 enzyme (cIAP1) in the presence of ATP, a decreasing amount of the cleaved caspase-3 or cleaved caspase-9 proteins was observed. This was observed with a Western blot using an antibody against each of the respective caspases. A corresponding increase in the various lengths of the caspases was obtained only in the presence of the ubiquitin ligase cIAP1, as shown in FIG. 4A. It is worth noting that the amount of pro-caspase-3 and pro-caspase-9 stayed the same, regardless of the presence of cIAP1. Taken together, these results indicate that only the activated caspase-3 and caspase-9 were the substrates of the cIAP1 E3 ligase, and cIAP1 catalyzed the conjugation of a poly-ubiquitin chain onto the two caspases. The unprocessed caspase-3 and caspase-9, however, could not be ubiquitinized by cIAP1. This suggested that the binding between the E3 ligase cIAP1 with active caspases was required for cIAP1-mediated caspase ubiquitination.

An in vitro assay for the Ub ligase activity of cIAP1 before and after cleavage is shown in FIGS. 4B and 4C. About 400 nM caspase-3 or caspase-9 was incubated with 200 nM cIAP1 for 2 hours at 30° C. in a 20-μl final reaction volume that contained 100 nM ubiquitin activating enzyme, 400 nM Ub conjugating enzyme Ubc H6 (E2), 20 μM ubiquitin, 2 mM Mg-ATP, 40 mM Tris-HCl (pH 7.5), and 50 mM NaCl. The reaction mixtures were subjected to SDS-PAGE and the resolved samples were transferred to a nitrocellulose filter. The ubiquitination of both caspase substrates was analyzed by Western blotting with an antibody against caspase-3 (lanes 1 and 2) or caspase-9 (lanes 3 and 4). Both caspase samples were a mixture of the pro-form and the active form. The asterisk (*) indicates the mono-ubiquitinated (Ub)₁ active caspase-9. The poly-uniquitinated caspase-3 and -9 are denoted by (Ub)n.

Using this assay system, both the full-length and Omi-cleaved cIAP1 were compared for their ubiquitin ligase activity on caspase-3 and caspase-9. When an increasing amount of both IAP molecules were tested, the unprocessed cIAP1 protein at 150 nM catalyzed the ubiquitin conjugation on both caspase-3 and caspase-9, as shown in FIGS. 4B and 4C, lane 5, respectively. The same amount of the Omi-cleaved cIAP1, in contrast, showed little E3 activity for caspase-3, shown in FIG. 4B, lane 9, and much weaker activity for caspase-9, shown in FIG. 4C, lane 9. It was concluded that the Omi cleaved IAP but could not promote ubiquitin activity.

### Example 7

To understand why cleaved cIAP1 manifests a lower activity in caspase inhibition and ubiquitin conjugation, the cleavage sites were mapped on cIAP1. About 5 μg of full-length cIAP1 (GST-fused) was incubated with 0.4 μg of Omi WT for 2 hours at 30° C. The cleaved cIAP1 sample is shown in FIG. 5, together with Omi (lane 2), the full-length cIAP1 alone (lane 1), and Omi alone (lane 3). The samples were subjected to electrophoresis on a 7.5-20% linear gradient gel. The resolved samples were transferred to a PVDF membrane followed by Coomassie Brilliant Blue R250 staining. Four cleavage polypeptide fragments (panel A, F1-F4) were generated and 10 pmol of each were excised and subjected to N-terminal sequencing by the Edman Degradation method. The two 30 kDa polypeptides in lane 2 are GST as determined by N-terminal sequencing. Several degraded polypeptide bands were already in the full-length cIAP1 preparation, such as that labeled with an asterisk (*). Amino acid sequencing confirmed that this band was a fragment of cIAP1 starting from Serine 147, and identical to the band appearing in the Omi-treated sample (labeled with an arrow plus an asterisk).

A map of Omi cleavage sites on human cIAP1 is shown in FIG. 5B. The domain structure of human cIAP1 is illustrated as follows: BIR1 domain is the first darkened area, BIR2 is the second darkened area, BIR3 is the 3rd darkened area, CARD is labeled, and RING zinc finger domain is labeled. The three underlined amino acid sequences were the amino terminal sequences (Edman Degradation) of the cleaved cIAP1 fragments F1/F2, F3 and F4, respectively. Omi cleaved cIAP1 after the residue Thr4, Asn133, and Leul61, as denoted by the three arrows.

The sequencing results showed that fragments F1 and F2 started with the same amino acid sequences of ASQRLFPG, indicating that the first cleavage site in cIAP1 molecule was after the residue Thr4 (the first arrow in FIG. 5B). The N-terminal sequencing showed that both fragments F3 and F4 started with GST, indicating that they are either GST or GST fused with partial cIAP1 fragment. The calculated molecular size between the amino terminum of GST and that of cIAP1 is about 27 kDa. This number is very close to the molecular size of fragments F3 and F4, although F3 is slightly smaller than F4. It is, therefore, very likely that F4 was the GST fused with a partial cIAP1 that ended at the first cleavage site (residue Thr4). F3 ended a bit more towards the N-terminal part of the fusion molecule. There is a thrombin cleavage site in the GST-cIAP1 construct. Since both Omi and thrombin are serine proteases, and they may share some common cleavage sites, one possibility is that the cleaving at the thrombin cleavage site by Omi produced F3.

Although the C-terminal sequence of each fragment is not known, taking into consideration both the size of each cleavage fragment and the three cleavage sites, it is likely that F1 contains residues 5-133. F2 is composed of residues 5-161. The stars (*) indicated a cleavage fragment already present in the GST-cIAP1 protein preparation, which was cleaved after residue Ser146 was identified by N-terminal sequencing. It was generated during the expression and purification of this GST fused cIAP1 by proteases in bacteria.

All three cleavage sites are located in the region that is N-terminal to the BIR2 domain of the molecule. There were two obvious outcomes as the result of this cleavage. The first outcome was that the BIR1 domain was removed from the whole molecule by the cleavage at the second and third sites. The rest of the molecule missing the BIR1 domain showed lower activity in caspase inhibition and ubiquitin conjugation, it suggested that although the BIR1 domain by itself did not inhibit caspases nor promoted ubiquitin conjugation, it might have some structural roles in allowing the whole molecule to perform its activity to the full scale.

The second outcome was the damaged linker region in the N-terminal of the BIR2 domain by the cleavage at the third cleavage site. The corresponding linker region in the BIR2 domain of XIAP is indispensable for its BIR2 to inhibit active caspase 3. If the same mechanism is true for the BIR2 linker in cIAP1, the third cleavage sites probably generated a deficient BIR2 linker and, thus, adversely affected its anti-caspase activity.

### Example 8

Omi cleaves cIAP1 in cells and this cleavage promotes caspase activation and etoposide-induced cell death. To ascertain the relevance of Omi cleavage of IAPs in cell death, IAPs were tested to determine whether they were, in fact, cleaved in cultured cells under apoptotic conditions. Human histiocytic lymphoma U937 cells (ATCC) at 1×10⁶ cells/ml were cultivated in an atmosphere of 5% CO₂ in air at 37° C. They were left untreated, treated with 100 μM etoposide, or treated with 2 μM staurosporine at 37° C. for 8 hours, 24 hours, and 48 hours. The cells were subsequently harvested and lysed with 0.5% CHAPS Buffer A supplemented with 1 mM DTT and protease inhibitors. The cell debris was removed by centrifugation for 20 minutes at 12,000×g. A total of 30 μg protein was resolved by 12% SDS-PAGE, followed by transferring to a nitrocellulose filter. The filter was probed with an antibody against cIAP1. The arrow indicated the full-length cIAP1 molecule, and the asterisk indicated an unrelated polypeptide band. As can be seen in FIG. 6, the IAP was completely cleaved. Treatment of U937 cells with etoposide or staurosporine for 24 hours led to complete cleavage of endogenous cIAP1, as shown by the disappearance of the fill-length cIAP1 molecules.

### Example 9

Related to Example 8, transient transfection assays were used to investigate whether cIAP1 cleavage was achieved by Omi in cell death induced by etoposide. 1.5 μg of either the c-Myc tagged FL Omi WT or the FL Omi SA expression construct was co-transfected with 1.5 μg N-terminal FLAG tagged cIAP1 construct into HEK 293 cells. After being transfected for 24 hours, the cells were treated with 100 μM etoposide for 24 hours to induce DNA damage and Omi release. The cells were harvested, and cell free extracts were prepared. A total of 40 μg cytosolic protein was separated on 12% SDS-PAGE, and the resolved samples were transferred to a nitrocellulose filter. The filter was subjected to Western blotting with a M2 FLAG antibody. The arrowhead indicates the cleavage product of cIAP1 upon etoposide treatment. The asterisk indicates the polypeptides unrelated to this etoposide treatment. Overexpression of cIAP1 and full-length Omi WT in HEK 293 cells resulted in cIAP1 cleavage exclusively in etoposide-treated cells (compare lane 3 with lane 4 in FIG. 6C). This etoposide-dependent cIAP1 cleavage was prevented in cells overexpressing the protease dead mutant Omi SA, as shown in FIG. 6C, lane 5. These results suggested that the cIAP1 cleavage was achieved by the serine protease Omi, which was released from mitochondria into the cytosol upon etoposide treatment (data not shown). A full-length Omi WT, as shown in FIG. 6C, but not the protease dead/inactive mutant Omi SA, cleaved cIAP1 in cells during etoposide-induced cell death.

The arrowhead in the middle and lower panels indicates the cleaved fragment of caspase-8 and caspase-3, respectively. This cIAP1 cleavage correlated with the occurrence of a cleaved fragment of caspase-8 and caspase-3 after etoposide treatment for 48 hours, shown in FIG. 6D, middle and bottom parts, lane 3. Overexpression of the protease dead mutant, AVPS Omi SA, greatly attenuated cIAP1 cleavage, shown in FIG. 6D, top part, lanes 4-6, and abolished capase-8 and caspase-3 cleavage. These results show that Omi cleaves cIAP1, and this IAP cleavage potentiates caspase activation in etoposide-induced cell death. The cytosolic form of Omi WT, but not the protease dead/inactive Omi SA, cleaved cIAP1 in cultured cells is shown in FIG. 6D.

### Example 10

The antibody materials used in the previous examples were as follows: Polyclonal antibody against cIAP1 was purchased from BIOCARTA (San Diego, Calif.). Polyclonal antibodies against cIAP2 and XIAP and the monoclonal antibody against human Survivin were purchased from R & D Systems (Minneapolis, Minn.). HRP conjugated anti-GST antibody, anti-c-Myc, and anti-FLAG M2 antibodies were purchased from Sigma (St. Louis, Mo.). Polyclonal antisera against Omi and Smac were obtained from rabbits immunized with purified recombinant Omi and Smac proteins by Rockland Immunochemicals Inc. (Gilbertsville, Pa.). HeLa cells were purchased from the National Cell Culture Center at Biovest International, Inc. in (Minneapolis, Minn.).

### Example 11

cDNA constructs previously used were generated using the following protocol. An EST clone for full length human Omi (Genbank accession number: AI979237; IMAGE clone number: 2493256) was obtained from Incyte Genomics (Palo Alto, Calif.) and used as the DNA template for subcloning. The cDNA for the mature form of Omi was PCR amplified and subcloned into the Nde I/Xho I sites of the pET21 b vector (Novagen, Madison, Wis.) to generate C-terminal hexa-His tagged constructs. The point mutation and various deletion mutations of Omi were generated by PCR, and the nucleic acid molecules were subcloned via the same restriction sites into pET21b. Human EST clones for both Livin α (Genbank Accession No. BC014475; IMAGE clone number: 4859588) and Livin β (Genbank Accession No. BG761924; IMAGE clone number: 4841724) were purchased from ATCC (Manassas, Va.). Both cDNAs were subcloned into the BamH I/Sal I sites of the pQE30 vector (QIAGEN, Valencia, Calif.) to generate N-terminal hexa-His tagged constructs.

Full-length human XIAP was subcloned by PCR via the BamH I/EcoR I sites into pGEX4T-2 (Amersham) using human XIAP EST clone (IMAGE: 5532247) as the cDNA template. The cDNA encoding the full-length human cIAP1 or cIAP2 was PCR amplified from a HeLa cDNA library and subcloned into the BamH I/EcoR I sites of the pGEX-4T-2 expression vector. The cDNA encoding the full-length Drosophila DIAP1 was PCR amplified using a template provided by Dr. Yigong Shi at Princeton University and subcloned into the BamH I/Not I sites of pGEX-4T-1 expression vector.

To generate N-terminal 3×FLAG tagged human cIAP1, the full-length cDNA was amplified and subcloned into the Xba I/BamH 1 sites of the p3×FLAG-CMV-7 expression vector (Sigma). For construction of C-terminal c-Myc (SEQ ID NO. 80) tagged mammalian Omi expression vectors, the cDNA encoding the full-length Omi was PCR amplified with the following primers: forward, SEQ ID NO. 81; reverse, SEQ ID NO. 82. The Xba I-Kpn I fragment was inserted into the pcDNA 3.1 (−) vector (Invitrogen, Carlsbad, Calif.) through Xba I-Kpn I sites. The vector for the mature form of Omi (starting from AVPS) was generated similarly, except that a different forward primer was used: forward, SEQ ID NO. 83. The S306→Ala mutants were generated by replacing the BamH I/EcoR I fragment with a fragment containing the corresponding mutated codon. The mutation-containing fragment was obtained by BamH I/EcoR I digestion of the pET 2 lb vector for Omi S306ΔAla.

All of these cDNA constructs were verified by DNA sequencing.

### Example 12

Expression and purification of recombinant proteins from bacteria was accomplished using the following protocol. The C-terminal hexa-His tagged wild-type and mutant Omi proteins were over-expressed in E. coli strain B121 (DE3) and purified with Ni-NTA Sepharose (QIAGEN, Valencia, Calif.) affinity chromatography. The N-terminal hexa-His tagged Livin α and Livin β were expressed in E. coli strain JM109, and the recombinant proteins were purified with Ni-NTA Sepharose affinity chromatography and further fractionated with Q-Sepharose ion exchange chromatography (Amersham). The GST-fusion forms of cIAP1, cIAP2, XIAP, and DIAP1 were expressed in E. coli strain B121 (DE3) and purified with Glutathione Sepharose affinity chromatography followed by Superdex 200 gel filtration chromatography (Amersham). The purity of proteins was checked by SDS-PAGE. The protein concentrations were determined by the modified Bradford method (Zor and Selinger, 1996).

### Example 13

The Omi/HtrA2 serine protease activity assay was conducted as follows. Proteins were incubated with wild-type or mutant Omi in PBST containing 20 mM Pi, pH 7.4, 100 mM NaCl, 0.5 mM EDTA, 0.05% Tween 20 and 1 mM DTT or in Buffer A containing 20 mM HEPES, pH 7.4, 10 mM KCl and 1.5 mM MgCl₂ for 2 hours at 30° C. or 37° C. The reaction mixture was resolved on SDS-PAGE and the cleavage results were monitored by Western Blotting using antibodies against the respective proteins, or Silver staining, or Coomassie blue staining.

### Example 14

The assay for caspase inhibitory activity of cIAP1 was as follows. In vitro translated, ³⁵S-labeled, procaspse-3 (1.5 μl) was mixed in a reaction volume for 40 minutes at 30° C. The reaction was carried out in the presence or absence of different IAP proteins and/or various forms of Omi protein. The reaction was stopped with the addition of 7 μl of 4×SDS sample loading buffer and the samples were separated on 15% SDS-PAGE and transferred to a nitrocellulose filter. The filter was exposed to a phosphor screen (Amersham) for one hour at room temperature. The in vitro transcription and translation of ³⁵S-labeled proscaspse-3 was carried out with the T_{N}T T7 coupled reticulocyte lysate system from Promega (Madison, Wis.). This His-tagged procaspase-3 was further purified with Ni-NTA Sepharose column before using.

### Example 15

The assay for ubiquitin Ligase activity of cIAP1 was as follows. The substrates caspase-3 and caspase-9 were incubated with varying concentrations of un-cleaved or Omi-cleaved cIAP1 in a 20-μl reaction volume that contained 40 mM Tris-HCl (pH 7.5), 50 mM NaCl, 100 nM ubiquitin activating enzyme (E1, Boston Biochem, Cambridge, Mass.), 400 nM Ubc H6 (E2, Boston Biochem), 20 μM of ubiquitin (Boston Biochem) and 2 mM Mg-ATP for 2 hours at 30° C. The reaction products were resolved on SDS-PAGE and transferred to a nitrocellulose filter. The ubiquitination of caspase substrates was analyzed by Western blotting using an antibody against caspase-3 and caspase-9 (R&D Systems).

### Example 16

Omi cleavage site mapping on cIAP1 by Edman degradation was as follows. About 51 g of cIAP1 was incubated with 0.4 μg of Omi at 30° C. under the reaction conditions previously described. The reaction products were resolved by SDS-PAGE and transferred to a PVDF membrane. About 10 pmol of each cIAP1 polypeptide fragments generated by Omi proteolysis was subjected to amino terminal sequencing by Edman degradation at the HHMI Biopolymer Laboratory/W. M. Keck Foundation at Yale University, New Haven, Conn.

### Example 17

Transfection of HEK 293 cells was accomplished using the following protocol. HEK 293 cells were plated in 6-well plates at 3-5×10⁵ cells/well and grown overnight prior to transfection in an atmosphere of 5% CO₂ in air at 37° C. The cells were transfected with a FLAG tagged cIAP1 expression construct and c-Myc tagged Omi expression constructs using Fugene 6 transfection reagent from Roche (Indianapolis, Ind.) according to the manufacturer's instructions. The cells were treated with 100 μM etoposide 24 hours after transfection and harvested 24 hours or 48 hours later. Cells were washed once in PBS and lysed by 0.5% CHAPS in Buffer A supplemented with 1 mM DTT and protease inhibitors. The cell lysates were centrifuged at 12,000×g for 20 minutes to remove the debris. The samples were then analyzed by Western blotting for Omi, cIAP1, caspase-3, and caspase-8. Electrophoresis and Western blotting technique included the following: the samples from in vitro activity assay or cell transfection were separated by SDS-PAGE followed by electrophoretic transfer onto the nitrocellulose filters. The filters were immunoblotted with appropriate antibodies, and the antibody detection was performed using a chemiluminescence detection kit from Perkin Elmer (Boston, Mass.).

### Example 18

A Biotin-Avidin Fab anti-idiotype liposome can be prepared for use in in vitro and in vivo anti-tumor systems. The liposomes contain an Omi family polypeptide for use in tumor suppression. The method can be initiated by producing monoclonal anti-idiotype antibody specific for B-Cell leukemia tumor-associated antigen B leukemia cells can be isolated from a patient and fused with K6H6/B5, a HAT sensitive heterohybridoma cell line. Serial two-fold dilutions of supernatants from fusion clones of hybridomas between patient B leukemia cells and the HAT-sensitive line are incubated overnight at 4° C. with microfiber plate wells, washed five times, and peroxidase-conjugated goat anti-human IgG, heavy and light chain, (TAGO, Burlingame, Calif.) is added for 3 hours, 37° C. Plates are washed and allowed to react with 5.5 mM ortho-phenylenediamine, 0.015% H₂O₂ (OPD) in citrate phosphate buffer, pH 5.0.

Positive clones secreting idiotype molecules in high concentration are selected, expanded, and later pooled. The idiotype molecules are purified from cell culture supernatants by immunoaffinity chromatography over anti-human IgG conjugated columns.

Next, monoclonal antibodies to the selected idiotype tumor-associated antigen can be produced. Purified idiotype from the patient B cell tumor are used to immunize Balb/c mice or C57/BL6 mice, in Freund's complete or incomplete, or mineral oil adjuvant. Three days after the final booster injection, isolated spleen cells are fused with the non-secreting mouse myeloma cell line, SP2/0 Ag 14 (ATCC Designation CRL 8287). Ten to fourteen days after fusion, hybridoma supernatants are screened for the presence of anti-idiotype antibody specificity by ELISA screening with the patient's B cell tumor idiotype. In these experiments, wells of a 96-well microtiter plate are coated with patient idiotype solutions, washed, then incubated with hybridoma supernatants containing anti-idiotype antibodies. After washing, peroxidase-conjugated goat anti-human IgG antibody (mouse immunoglobulin adsorbed) are added, and plates are developed with OPD as previously described.

Those clones that are reactive against patient B leukemia cells and not against unrelated B leukemia cells, benign lymphoid hyperplasic cells, normal human blood cells, and normal human serum are selected for further expansion and processing.

The reactive clones are selected and biotinylated phospholipids are prepared. Biotinylated phospholipids are prepared by dissolving phosphatidylethanolamine (PE, 5.1 mg) or phosphatidylserine (PS, 3.9 mg) in a solution (170 μl for PE; 130 μl for PS) of chloroformmethanol (2:1) with biotinyl N-hydroxysuccinimide ester (BNHS, 3.3 mg) (Sigma Chemicals, St. Louis, Mo.). 101l is added of a chloroform solution containing 15% (v/v) triethylamine. After a 2 hr incubation of the reaction mixture at ambient room temperature (18° C.), the crude mixture is stored at −70° C.

The crude biotinylated lipid is then purified by high-performance liquid chromatography (HPLC) using a Waters system (Waters Associates, Milford, Mass.) with two solvent delivery units (M-45 and Model 510) and a Model 680 gradient controller.

Biotinylated liposomes are then prepared. Biotinylated phospholipids (BPE or BPS) are dissolved in chloroform/methanol (2:1) and molar equivalents of each corresponding lipid (BPE or BPS) are added to 12 mm×75 mm glass tubes to yield the final percentage of biotinylated lipid desired (e.g., 5, 10, 20%). Concentrations of 0.01 to 1 mol % of total lipid are achieved.

To prepare liposomes, the biotinylated lipid/native lipid mixture (e.g., 2 μmol of the stock lipid mixture in chloroform/methanol) is evaporated to dryness under a stream of nitrogen and then placed in a vacuum dessicator overnight. The lipid is resuspended by syringe injection (e.g., 50 μl lipid in chloroform/methanol into 1.0 ml PBS) in a final concentration of 1 mg/ml in PBS, pH 7.2-7.4, then sonicated under nitrogen in an ice-cooled chamber for 10 min in a Branson-Sonifier® Model 130 (Branson Ultrasonics Corporation, Danbury, Conn.). The resulting suspension is centrifuged at 10,000 rpm for 20 min, and the biotinylated liposomes in the supernatant fraction used within 24 hr after preparation.

To encapsulate Omi family polypeptide molecules, the biotinylated lipid/native mixture is resuspended by injection (e.g., 50 μl lipid in chloroform/methanol into 1.0 ml PBS) into an Omi family polypeptide containing PBS solution. After sonication and centrifugation at 10,000 rpm for 20 min, Omi-biotinylated liposomes are purified as such, liposome preparations are centrifuged at 13,000×g in a microcentrifuge, pelleted liposomes are washed with PBS, and pelleted liposome fractions are resuspended in PBS buffer for use.

Once the liposomes are prepared, Fab fragments of anti-idiotype antibodies must be prepared. After purification of anti-idiotype IgG antibody, Fab fragments are prepared by papain cleavage.

Biotinylated Fab fragments of anti-tumor associated idiotype antibodies are obtained by using an N-hydroxysuccinimidobiotin (NHS-Biotin) (Sigma Chemical). In this method, 2 mg of Fab fragments are dissolved in 1 ml of sodium phosphate buffer (PBS), pH 7.5-8.5, in a 16×125 mm test tube. Immediately before use, 1 mg of NHS-Biotin is dissolved in 1 ml dimethylformamide (DMF). 75 μl of the dissolved NHS-Biotin is added to the Fab containing test tube. The tube is incubated on ice (4° C.) for 2 hrs. The unreacted biotin may be removed by dialysis (e.g., Slide-A-Lyzer Dialysis Cassette) or with a D-Salting Column (Pierce Chemical, Rockford, Ill.). Alternatively, unreacted biotin may be removed by centrifugation of the product at 1000×g for 15-30 min using a microconcentrator. After centrifugation, the sample is diluted in 0.1 M sodium phosphate, pH 7.0. The process can then be repeated twice more. The biotinylated protein may be stored at 4° C. in 0.05% sodium azide prior to use.

Finally, Fab-Omi liposomes utilizing biotinylated Fab molecules, biotinylated liposomes and avidin are prepared. The biotinylated Fab fragments in PBS are mixed with a twenty-fold molar excess of egg white avidin (Vector Labs, Burlingame, Calif.; Sigma Chemical, St. Louis, Mo.), incubated overnight at 4° C. The excess avidin is removed by passage of the mixture over anti-human light chain affinity columns (e.g., Pharmacia Sepharose 4B). Fab-biotin-avidin molecules are eluted with citrate buffer, pH of then pooled fractions are dialyzed against PBS, pH=7.0. A suspension of biotinylated Omi mutant protein (Omi)-containing liposomes is mixed with Fab-biotin-avidin solutions in PBS to yield avidin to free biotin ratios on the liposome surfaces of approximately 2:1, 5:1, 10:1, and 20:1 molar ratios. After incubation overnight at 4° C. on a rotational shaker, liposomes are passed through a Pharmacia Sephadex G200 column. The Fab-Omi liposomes are collected in the void volume and resuspended in PBS. The resultant Fab-Omi liposomes are available for use.

### Example 19

The present Example relates to in vitro killing of idiotype-bearing human B leukemia cells by anti-idiotype-specific Fab-biotin liposomes, which were prepared in Example 18.

Anti-idiotype Fab-Omi Liposomes with radiolabeled target idiotype-bearing B leukemia cells. The anti-idiotype Fab-armed liposomes containing Omi protein can be mixed with Cr-51-labeled idiotype-bearing B leukemia target cells according to the following procedure: Serial two-fold dilutions of anti-idiotype Fab-Omi-containing liposomes are made in RPMI medium to yield a final liposome-target ratio of 400:1, 200:1, 100:1, 50:1, 25:1, and 12.5:1. 100 μl/well of each of the above liposome dilutions are placed in two sets of triplicate wells of a 96 well round-bottomed microtiter plate. To one set of triplicate anti-idiotype Fab-armed liposome wells is added 100 μl of radiolabelled target cells. To a second set of triplicate anti-BSA liposome wells is added 100 μl of radiolabelled target cells (first negative liposome control). To a third set of triplicate anti-idiotype liposome wells is added 100 μl of unrelated idiotype-bearing B leukemia cells (second negative liposome control).

To a fourth set of triplicate wells is added 100 μl labeled target cells and 100 μl of 5% Triton-X100 detergent to yield maximally releasable counts as a positive control. To a fifth set of triplicate wells is added 100 μ/well of radiolabelled target cells and 1001 μl of RPMI medium alone as a spontaneous release control. The microtiter plate is then covered and centrifuged for 3 min at 200×g, then incubated for 4 hr at 37° C. in a 5% CO₂ incubator. After incubation 50-100 μl of supernatant is aspirated from wells without disturbing the cell pellet and transferred to counting tubes. Tubes are then placed in a gamma emission counter to assay the Cr-51 release using 1 min counting time per sample tube. Percent specific target cell lysis for each liposome:target cell ratio is calculated by measuring the triplicate counts per minute (cpm) values for each liposome:target cell ratio and for the spontaneous and maximum release wells.

### Example 20

The present example relates to the inhibition of in vitro proteolysis of IAP substrates by the Omi-derived family of polypeptide inhibitory molecules. As shown, AVPS serves as the IAP binding motif required for the Omi molecule's catalytic activity in hydrolyzing its IAP substrates. Omi-derived inhibitory polypeptide molecule variants can be made utilizing the QuickChange site-directed mutagenesis kit (Stratagene, La Jolla, Calif.) to induce point mutations in the Omi nucleic acid sequence resulting in the substitution of single or multiple amino acids in the Omi variant catalytic site polypeptide sequence. Thus, Omi-derived inhibitory molecules with the polypeptide sequences can be made from their corresponding nucleic acid sequences.

Inactive Omi variant polypeptides, containing AVPS moieties, are anticipated to inhibit the IAP-cleaving action of wild-type Omi polypeptide where the variant to wild-type ratios range between 10:1 to 100:1, perhaps through steric hindrance or competitive inhibition principles. Through steric hindrance, the AVPS-Omi inactive polypeptide molecule binds to LAP and prevents binding of wild-type Omi molecules, thereby blocking IAP cleavage by Omi wild-type molecules. In competitive inhibition modes of action, the AVPS-Omi inactive polypeptide competes for binding to IAP by wild-type Omi through the AVPS moiety wherein IAP cleavage is inhibited because relatively few IAP molecules are bound to wild-type Omi.

### Example 21

In this example, kits for detection and quantitation of Omi wild-type and mutant polypeptides and fragments are made. Antigens to be prepared for immunization and to be used as standards in immunoassays include Omi family members. Both Omi-derived polypeptide and nucleic acid antigens are prepared and were previously described.

Goat and rabbit polyclonal antibodies and mouse monoclonal antibodies to the Omi-derived wild-type and mutant polypeptide and nucleic acid molecules are prepared by methods that are known to those of skill in the art. Once monoclonal and polyclonal antibodies to Omi-derived polypeptide and nucleic acid molecules have been made, they can be utilized in immunodiagnostics kit assays for the detection and quantitation of the Omi-derived molecules.

For example, a sandwich enzyme immunoassay (EIA) can be utilized in a microtiter plate format according to the following procedure: Wells of EIA microtiter plates are coated with 100 μl/well of 10 ug/ml goat polyclonal antibody against Omi whole molecule polypeptide in carbonate buffer (0.75 g sodium carbonate, 1.43 g. sodium bicarbonate, QS to 500 ml) for 1-2 hr at 20° C. or overnight at 2-8° C. The coating fluid is aspirated, and the plate wells are blocked with 200 to 250 μl/well of 1% BSA in PBS buffer, pH 7.4 for 2-4 hr at 37° C. or overnight at 2°-8° C. The plates are washed three times with >250 μl/well of PBS, 0.05% Tween 20 detergent, 0.05% sodium azide. The plates are dried and covered with sealing tape, and stored at 2°-8° C. Add 200 μl of Omi polypeptide containing cell extract samples (5-10 μg/ml in PBS-Tween) is added into each of triplicate wells in row 1 of the microtiter plate. 100 μl of PBS-Tween is placed into triplicate wells of rows 2-12. The Omi WT or mutant polypeptide is serially diluted into PBS-Tween by taking 100 μl/well in row 1, depositing it into corresponding wells of row 2, mixing, then proceeding similarly down through row 12. Incubate for 1 hr at 18°-22° C. Wash plate wells three times with PBS-Tween. A murine monoclonal antibody to the Omi variant polypeptide molecules (e.g., antibody to PDZ, AVPS, or hinge regions), starting in triplicate wells in row 1 with 5 μg/ml in PBS-Tween, then serially diluted two-fold through row 12 as previously. The mixture is incubated for 1 hr, 18°-22° C. Aspirate and wash plate wells three times with PBS-Tween. Add DAKO horseradish peroxidase (HRP)-conjugated goat antimouse IgG antibody to wells (100 μl/well, 1:100 dilution). (DAKO Corporation, Carpinteria, Calif.) Incubate 1 hr, 18°-22° C. Wash three times with PBS-Tween as above. Add 100 μl/well of ortho-phenylenediamine (OPD) substrate solution, containing H₂O₂, for 15 min. Stop reaction with 100 μl/well of 2N sulfuric acid, and read plates at 490 nm.

Similarly, a competitive enzyme immunoassay in microtiter plate format may be produced for detection and quantitation of Omi variant polypeptides and nucleic acids by coating plate wells with anti-Omi antibodies of varying specificities (e.g., anti-AVPS, anti-PDZ, antihinge), then incubating with both (1) HRP-labeled Omi variant molecules (e.g., Omi whole molecule, OmiΔPDZ/Hinge/AVPS) (e.g., 50 μl/well) and (2) serial dilutions of samples containing Omi variant molecules (e.g., AVPS, hinge, etc.) (e.g., 501 μl/well). Competitive EIA plate wells are developed by the previously described procedures for sandwich assays.

### Example 22

In this example, hybridization kits are described for the detection of Omi wild-type and Omi variant nucleic acid sequences. Omi wild-type and variant nucleic acid sequence molecules are prepared by either PCR methodology. DNA or RNA primers are prepared containing desired probe sequences. For example, a probe can be prepared from between 5 to 100 bp in length for the PDZ region to detect PDZ nucleic acid sequences. Similarly, probes can be prepared for active Omi catalytic triad variants, shortened Omi molecule variants, and inactive Omi molecule variants. Promoter sequences can be added to DNA or RNA probes as has been described in the art.

Omi WT molecule and Omi variant cDNA synthesis and Cy3/Cy5 labeling is as follows: Heat 10-15 ug Omi sample RNA with 1.7 μl random primers (3 ug/ul; Invitrogen Cat. No. 48190-011) and 15.9 μl H₂O at 70° C. for 10 min. Snap cool on ice and centrifuge. To each reaction tube, add 1.5 μl of Cy3-dCTP (Amersham Pharmacia Biotech Cat. No. PA53021) with 1.0 μl H₂O or 2.5 μl of Cy5-dCTP (Amersham Cat No. PA55021). Add 11.6 μl of Master mix as follows: 6 μl of 5×First Strand Buffer, 3 μl of DTT (100 mM), 0.6 μl of dNTPs (25 mM each dA/G/TTP, 10 mM dCTP), 2 μl of SuperScript II (200 U/ul; Invitrogen Cat. No. 18064-014). Incubate reaction at 25° C. for 10 min followed by 42° C. for 110 min.

Briefly centrifuge the labeling reaction tubes. Add 10 μl 1N NaOH and heat at 70° C. for 10 min to hydrolyze the RNA. Briefly centrifuge and neutralize by adding 10 μl 1N HCl. Using the MinElute PCr purification kit (Qiagen Cat. No. 28004), combine Cy3 and Cy5 labeled cDNA samples in a single Eppendorf tube and add 500 μl Buffer PB. Apply to MinElute column in collection tube and centrifuge at 13,000 rpm for 1 min. Purple coloration of the membrane indicates efficient labeling of both cDNA samples. Discard flow-through and place MinElute column back into the same collection tube. Add 50 μl Buffer PE to MinElute column and centrifuge at 13,000 rpm for 2 min to dry the membrane. Carefully transfer the MinElute column into a fresh 1.5 ml tube A. Add 10 μl MilliQ H₂O pH 7-8.5 carefully to the center of the membrane and allow to stand for 1 min. Centrifuge at 13,000 rpm for 1 min to collect cDNA (yield ˜80%). Place the MinElute column into a fresh 1.5 ml tube B. Add 5 μl MilliQ H₂O pH 7-8.5 to the center of the membrane and allow to stand for 1 min. Centrifuge at 13,000 rpm for 1 min to collect residual cDNA. Transfer 4.5 μl from tube B to tube A (final volume 14.5 μl).

For hybridization, the following procedure is used: Mix purified Cy3/Cy5 sample with hybridization solution (14.5 μl of Cy3/Cy5 labeled cDNA, 3 μl filtered 20×SSC, 2.5 μl of filtered 2×SDS). Prepare a slide heating block at setting 7 (Scientific Laboratory Supplies Cat. No. MIC 4302). Preheat the hybridization chamber (CMT-Hybridization Chambers (5), Corning Cat. No. 2551). Heat hybridization solution at 99° C. for 2 min to denature cDNA. In the meantime, prepare the slide and a 24×24 mm coverslip. Blow the slide gently with an air-duster (Dust-Pro PressurizEd Duster, Sigma Cat. No. Z37,952-2) to remove dust and pre-heat it on the heater. Use the duster to remove any dust from the coverslip. Position the coverslip ready to pick up. When ready, immediately centrifuge the hybridization solution briefly, put the slide into the chamber, pipet 30 μl 3×SSC into each of the two wells of the chamber, and apply the solution onto the slide at the edge of the spotted area avoiding bubble formation by using curvededge fine forceps to set the coverslip in place. Close the chamber and immerse it in a 63° C. waterbath. Incubate chambers overnight (16-20 hr).

Prepare stock wash solutions, Wash A (0.5×SSC/0.01% SDS) and Wash B (0.06×SSC). Transfer slides one at a time from the chamber to the Coplin jar containing Wash A and let the coverslip fall off by gently moving the slide vertically in the solution. Once the coverslip is removed, transfer the slide quickly to the rack in the trough of Wash A. Continue with coverslip removal for the next slide. When all slides are on the rack, wash by vigorous agitation for 5 min at room temperature. Transfer the slides quickly to the rack in the second trough containing Wash B. Wash by vigorous agitation for 3 min at room temperature. Transfer the rack to the third trough containing Wash B and wash by vigorous agitation for 3 min at room temperature. Dry slides and store in a slide box until scanning.

The ScanArray Express (Perkin Elmer Life Sciences, Boston, Mass.) can be used to scan the slides. Alternatively, the the Image Trak Epi-Fluorescence System (Perkin Elmer Life Sciences, Boston, Mass.) can be used for 96, 384, or 1536 well plates.

### Example 23

It is believed that elimination of endogenous Omi makes cells more resistant to apoptosis. To prove this small interfering RNA (siRNA) was used to eliminate Omi expression. siRNA oligonucleotides against Omi (si-Omi) were transfected twice into HeLa cells with Luciferase GL, with the siRNA duplex as a control (Ctrl). All of the siRNA-transfected samples were treated with 2 μM proteasome inhibitor MG132 before being subjected to 250 ng/mL TRAIL treatment for 4 hr. MG132 was used to block proteasome-mediated cIAP1 degradation.

Ten micrograms of protein per sample were subjected to immunoblotting for endogenous Omi and cIAP1. Immunoblotting for Actin was to show equal sample loadings. The three immunoblotting results were obtained from the same filter.

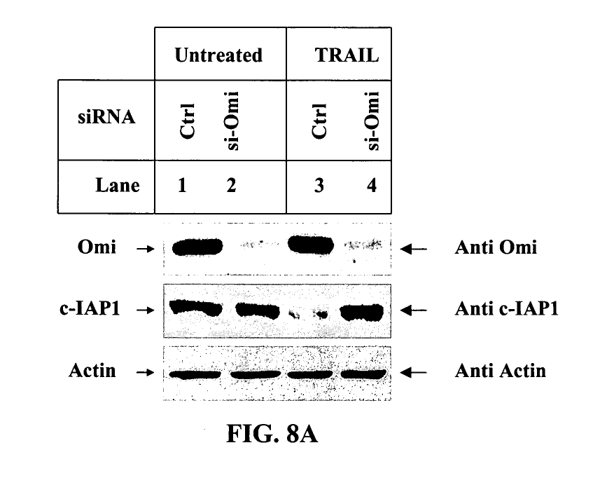

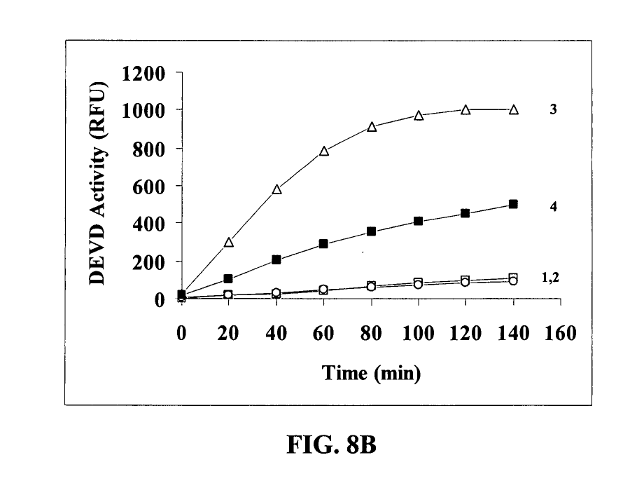

Transfection of cells with small interfering RNA (siRNA) molecules against Omi (si-Omi) effectively eliminated Omi protein expression, as shown in FIG. 8A. The corresponding cIAP1 cleavage under TRAIL treatment was reduced, as shown at FIG. 8A, middle, lane 4. The same results were obtained for cIAP2 and XIAP (data not shown). This reduced IAP cleavage correlated with a two- to approximately three-fold lower caspase activity in TRAIL-treated cells (FIG. 8B, curves 3, 4). Taken together, Omi cleavage of IAPs happens directly in apoptotic cells and represents an important step in Omi-mediated apoptotic progression.

### Example 24

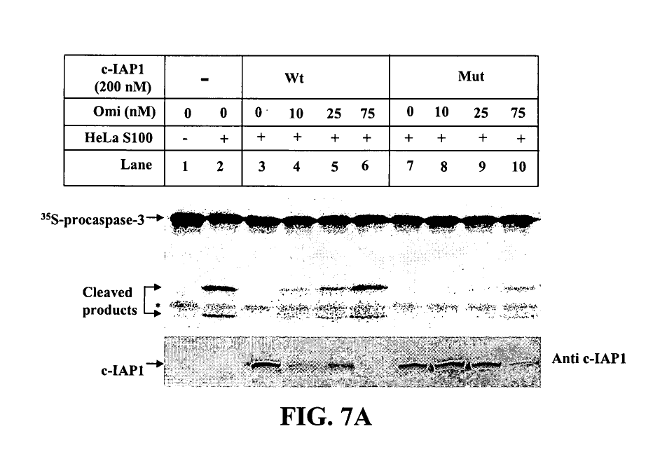

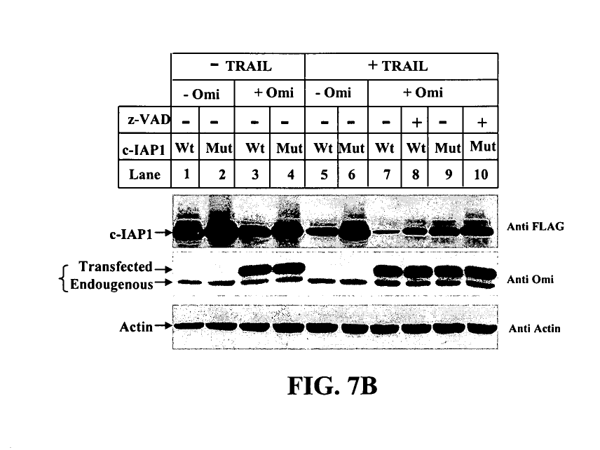

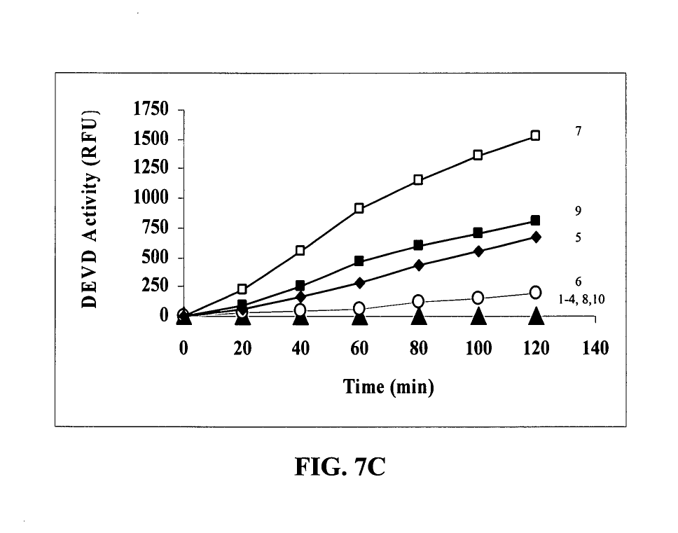

Mutant cIAP1 is more resistant to Omi cleavage and better protects cells from apoptosis. To test this theory, recombinant cIAP1 protein was mutated at the three preferred cleavage sites. The mutated IAP was tested, whereby its activity in vitro was assayed. The mutant cIAP1 protein maintained anti-capase activity (FIG. 7A, top, lane 7); this caspase inhibitory activity, however, could not be relieved by low concentrations of Omi (FIG. 7A, lop, lanes 8, 9) due to its resistance to Omi cleavage (FIG. 7A, bottom, lanes 8, 9). In contrast, Omi at the same concentration already cleaved wild-type cIAP1 and reactivated caspases (FIG. 7A, lanes 4, 5). Omi at 75 or 10 nM resulted in similar amount IAP cleavage and caspase reactivation for mutant and wild-type cIAP1, respectively (FIG. 7A, lanes 10, 4), indicating that mutant cIAP1 is ˜7.5-fold more resistant to Omi protease.

Further testing was done to determine whether this mutant cIAP1 could better block caspase activity in HeLa cells by transfection assays. Overexpression of the cytosolic form of Omi, MAVPS Omi, mimics the already released Omi upon certain apoptotic treatment, and thus, could distinguish the proapoptotic effect of Omi from other apoptotic factors released from mitochondria. The first methionine was likely removed after it was expressed in transfected HeLa cells, as it could bind to cIAP1 in a pull-down assay (data not shown). Overexpression of MAVPS Omi led to wild-type cIAP1 cleavage independent of apoptotic stimulus (FIG. 7B, top, cf. Lanes 3 and 1), whereas, mutant cIAP1 was more resistant (FIG. 7B, top, cf. Lanes 4 and 2). Under TRAIL treatment, and in the absence of exogenously transfected Omi, the mutant cIAP1 was more resistant than the wild type to the relatively limited amount of endogenous Omi (FIG. 6B, cf. Lanes 5 and 6). This cleavage resistance correlated with a threefold reduced DEVD activity in TRAIL-induced apoptotic cells (FIG. 7C, curves 5, 6). When more Omi was present by transfection, mutant cIAP1 was still more resistant to Omi (FIG. 7B, top, cf. Lanes 7 and 9), and this also correlated with a better caspase inhibition (FIG. 7C, cf. Curves 7 and 9). In addition, the majority of cIAP1 cleavage was not caspases, because it could not be inhibited by the caspase inhibitor z-VAD-fmk (FIG. 7B, top, lanes 8, 10), which was consistent with the results that Omi did not degrade caspases in vitro. The mutant cIAP1 was not absolutely resistant to Omi, because cleavage could also occur at other sites. Nonetheless, this mutant cIAP1 already manifested resistance to Omi cleavage and better-inhibited caspases, demonstrating that IAP cleavage by Omi plays an important role in apoptotic progression.

As such, cleavage-site mutant cIAP1 makes cells more resistant to apoptosis. The wild-type or mutant cIAP1 proteins at 400 nM were preincubated with Omi for 2 h at 37° C. in 10 μL of Buffer A and assayed for their caspase inhibitory activity in HeLa S100 extracts supplemented with DATP and cytochrome c. The caspase-3 cleavage activity was detected on a PhosphorImager (top panel), and cleavage of cIAP1 was detected on the same filter by an anti-GST antibody (bottom panel). A total of 750 ng of WT or mutant cIAP1 in p3×flag-CMV-7 construct was transfected into HeLa cells with or without cotransfection of 100 ng MAVPS Omi-Myc in pcDNA3.1 construct for 3 h using Lipofectamine Plus Reagent. The transfected cells were left untreated (lanes 1-4) or treated with 100 ng/mL TRAIL for 4 h (lanes 5-10). A total of 100 μM z-VAD-fmk was added to the culture medium 2 h before TRAIL treatment (lanes 8, 10). Total cell extracts were made and 20 μg of protein per sample was analyzed by SDS-PAGE and Western blotting. CIAP1 was detected with an anti-Flag antibody (top panel). Omi was detected with a polyclonal antibody (middle panel) so that both the endogenous (lower band) and exogenously expressed (upper band) Myc-tagged Omi were detected. Immuno-blotting for Actin was to show equal sample loadings (bottom panel). The three immunoblotting results were obtained from the same filter. The DEVD activity for the samples in B. The number next to each curve represents the DEVD activity for the same numbered samples in B. The curve that lies on the X-axis (▴) is the DEVD activity for samples in lanes 1-4, 8 and 10 in B. DEVD relates to a fluorogenis substrate used to measure activity of caspase.

Thus, there has been shown and described a method and composition for cleaving IAPs which fulfills all the objects and advantages sought therefor. It is apparent to those skilled in the art, however, that many changes, variations, modifications, and other uses and applications to the method and composition for cleaving IAPs are possible, and also such changes, variations, modifications, and other uses and applications which do not depart from the spirit and scope of the invention are deemed to be covered by the invention, which is limited only by the claims which follow.

### Reference List

Chai, J., Du, C., Wu, J. W., Kyin, S., Wang, X., and Shi, Y. (2000). Structural and biochemical basis of apoptotic activation by Smac/DIABLO. Nature 406, 855-862.
Chai, J., Shiozaki, E., Srinivasula, S. M., Wu, Q., Datta, P., Alnenri, E. S., Shi, Y., and Dataa, P. (2001). Structural basis of caspase-7 inhibition by XIAP. Cell 104, 769-780.
Carrol, W. L., et al., J. Immunol. Methods 89:61 (1986).
Cryns, V., and Yuan, J. (1998). Proteases to die for. Genes Dev 12, 1551-1570. Du, C., Fang, M., Li, Y., Li, L., and Wang, X. (2000). Smac, a mitochondrial protein that promotes cytochrome c-dependent caspase activation by eliminating IAP inhibition. Cell 102, 33-42.
Current Topics in Microbiology and Immunology 152: 183-193 (Bosma, M. J., Schuler, W. eds.)
Enari et al., 1998 Nature 391, 43-50.
Faccio, L., Fusco, C., Chen, A., Martinotti, S., Bonventre, J. V., and Zervos, A. S. (2000). Characterization of a novel human serine protease that has extensive homology to bacterial heat shock endoprotease HtrA and is regulated by kidney ischemia. J Biol Chem 275, 2581-2588.
Gray, C. W., Ward, R. V., Karran, E., Turconi, S., Rowles, A., Viglienghi, D., Southan, C., Barton, A., Fantom, K. G., West, A., et al. (2000). Characterization of human HtrA2, a novel serine protease involved in the mammalian cellular stress response. Eur J Biochem 267, 5699-5710.
Green, D. R. (2000). Apoptotic pathways: paper wraps stone blunts scissors. Cell 102, 1-4.
Hegde, R., Srinivasula, S. M., Zhang, Z., Wassell, R., Mukattash, R., Cilenti, L., DuBois, G., Lazebnik, Y., Zervos, A. S., Femandes-Alnerni, T., and Alnenri, E. S. (2002). Identification of Omi/HtrA2 as a mitochondrial apoptotic serine protease that disrupts inhibitor of apoptosis protein-caspase interaction. J Biol Chem 277, 432-438.
Holley, C. L., Olson, M. R., Colon-Ramos, D. A., and Kombluth, S. (2002). Reaper eliminates IAP proteins through stimulated IAP degradation and generalized translation inhibition. Nat Cell Biol 4, 439-444.
Hsueh, R. and Roach, T., Characterization of Cells by Flow Cytometry, Version 1.
Huang, D. C., and Strasser, A. (2000). BH3-Only proteins-essential initiators of apoptotic cell death. Cell 103, 839-842.
Huang, H., Joazeiro, C. A., Bonfoco, E., Kamada, S., Leverson, J. D., and Hunter, T. (2000). The inhibitor of apoptosis, cIAP2, functions as a ubizuitin-protein ligase and promotes in vitro monoubiquitination of caspases 3 and 7. J Biol Chem 275, 26661-26664.
Huang, Y., Park, Y. C., Rich, R. L., Segal, D., Myszka, D. G., and Wu, H. (2001). Structural basis of caspase inhibition by XIAP: differential roles of the linker versus the BIR domain. Cell 104, 781-790.
Lazcbnik, et al., 1995 Proc. Natl. Acad. Sci. USA 92, 9042-9046.
Li, F., Ambrosini, G., Chu, E. Y., Plescia, J., Tognin, S., Marchisio, P. C., and Altieri, D. C. (1998). Control of apoptosis and mitotic spindle checkpoint by survivin. Nature 396, 580-584.
Li, W., Srinivasula, S. M., Chai, J., Li, P., Wu, J. W., Zhang, Z, Alnemri, E. S., and Shi, Y. (2002). Structural insights into the pro-apoptotic function of mitochondrial serine protease HtrA2/Omi. Nat Struct Biol 9, 436-441.
Liu, X., Kim, C. N., Yang, J., Jemmerson R., and Wang, X. (1996). Induction of apoptotic program in cell-free extracts: requirement for dATP and cytochrome c. Cell 86, 147-157.
Liu et al., 1997 Cell 89, 175-184.
Locksley, R. M., Killeen, N., and Lenardo, M. J. (2001). The TNF and TNF receptor superfamilies: integrating mammalian biology. Cell 104, 487-501.
Martin et al., 1996 J. Biol. Chem. 2.
Martins, L. M., laccarino, I., Tenev, T., Gschmeissner, S., Totty, N. F., Lemoine, N. R., Savopoulos, J., Gray, C. W., Creasy, C. L., Dingwall, C., and Downward, J. (2002). The serine protease Omi/HtrA2 regulates apoptosis by binding XIAP through a reaper-like motif. J Biol Chem 277, 439-444.
Miller, L. K. (1999). An exegesis of IAPs: salvation and surprises from BIR motifs. Trends Cell Biol 9, 323-328.
Nagata, S. (1997). Apoptosis by death factor. Cell 88, 355-365.
Pickart, C. M. (2001). Mechanisms underlying ubiquitination. Annu Rev Biochem 70, 503-533.
Rathmell, J. C., and Thompson, C. B. (1999). The central effectors of cell death in the immune system. Annu Rev Immunol 17, 781-828.
Sahara et al., 1999 Nature 401, 168-173.
Salvesen, G. S., and Duckett, C. S. (2002). Apoptosis: LAP proteins: blocking the road to death's door. Nat Rev Mol Cell Biol 3, 401-410.
Samoszuk, M. K., et al., Hybridoma 6:605 (1987)
Shi, Y. (2002). A conserved tetrapeptide motif: Protentiating apoptosis through IAP-binding. Cell Death and Differentiation 9, 93-95.
Srinivasula, S. M., Hegde, R., Saleh, A., Datta, P., Shiozaki, E., Chai, J., Lee, R. A., Robbins, P. D., Femandes-Alnemri, T., Shi, Y., and Alnemri, E. S. (2001). A conserved XIAP-interaction motif in caspase-9 and Smac/DIABLO regulates caspase activity and apoptosis. Nature 410, 112-116.
Suzuki, Y., Imai, Y., Nakayama, H., Takahashi, K., Takio, K., and Takahashi, R. (2001). A serine protease, HtrA2, is released from the mitochondria and interacts with XIAP, inducing cell death. Mol Cell 8, 613-621.
Thornberry, N. A., and Lazebnik, Y. (1998). Caspases: enemies within. Science 281, 1312-1316.
Thielmans, K., et al., J. Immunol. 133:495 (1984).
van Loo, G., van Gurp, M., Depuydt, B., Srinivasula, S. M., Rodriguez, I., Alnemri, E. S., Gevaert, K., Vandekerckhove, J., Declercq, W., and Vandenabeele, P. (2002). The serine protease Omi/HtrA2 is released from mitochondria during apoptosis. Omi interacts with caspase-inhibitor XIAP and induces enhanced caspase activity. Cell Death Differ 9, 20-26.
Vaskovsky, V. E. and Kostetsky, E. Y., Lipid Res. 9:396 (1968).
Verhagen, A. M., Coulson, E. J., and Vaux, D. L. (2001). Inhibitor of apoptosis proteins and their relatives: IAPs and other BIRPs. Genome Biol 2, REVIEWS3009.
Verhagen, A. M., Ekert, P. G., Pakusch, M., Silke, J., Connolly, L. M., Reid, G. E., Moritz, R. L., Simpson, R. J., and Vaux, D. L. (2000). Identification of DIABLO, a mammalian protein that promotes apoptosis by binding to and antagonizing IAP proteins. Cell 102, 43-53.
Verhagen, A. M., Silke, J., Ekert, P. G., Pakusch, M., Kaufmann, H., Connolly, L. M., Day, C. L., Tikoo, A., Burke, R., Wrobel, C., et al. (2002). HtrA2 promotes cell death through its serine protease activity and its ability to antagonize inhibitor of apoptosis proteins. J Biol Chem 277, 445-454.
Wang, X. (2001). The expanding role of mitochondria in apoptosis. Genes Dev 15, 2922-2933.
Wu, G., Chai, J., Suber, T. L., Wu, J. W., Du, C., Wang, X., and Shi, Y. (2000). Structural basis of IAP recognition by Smac/DLABLO. Nature 408, 1008-1012.
Wu, J. W., Cocina, A. E., Chai, J., Hay, B. A., and Shi, Y. (2001). Structural analysis of a functional DIAP1 fragment bound to grim and hid peptides. Mol Cell 8, 95-104.
Yang, Y., Fang, S., Jensen, J. P., Weissman, A. M., and Ashwell, J. D. (2000). Ubiquitin protein ligase activity of IAPs and their degradation in proteasomes in response to apoptotic stimuli. Science 288, 874-877.
Zor, T., and Selinger, Z. (1996). Linearization of the Bradford protein assay increases its sensitivity: theoretical and experimental studies. Anal Biochem 236, 302-308.

1. A method for cleaving IAP, wherein the method comprises:
contacting in vitro, isolated IAP with an amount of an isolated Omi family polypeptide, whereby upon contact, the Omi family polypeptide will cleave the IAP.

2. The method of claim 1, wherein the IAP is selected from the group consisting of cIAP1, cIAP2, XIAP, Livin α, Livin β, and DIAP1.

3. The method of claim 1, wherein the Omi family polypeptide is selected from the group consisting of SEQ ID NOs. 44, 45, 48, 49, 54-57, 60-63, 66-75, and homologs thereof.

4. The method of claim 1, wherein the Omi family polypeptide to IAP molar ratio in vitro is equal to between 1:5 to 1:30 molar ratio of Omi to IAP.

5. The method of claim 1, wherein the Omi family polypeptide is expressed by a nucleic acid sequence molecule selected from the group consisting of SEQ ID NOs. 1-3,6-8, 11-19, 22-27, and 30-39, and homologs thereof.

6. The method of claim 1, wherein the in vitro conditions incubation time is 2 hours at 37° C. in solution.

7. A method for cleaving IAP, wherein the method comprises:
(a) isolating a population of cells, whereby the caspase found in the cells is bound by IAP;
(b) contacting in vitro the isolated cell population with the Omi family polypeptide whereby upon contact, the Omi family polypeptide cleaves the IAP from the caspase.

8. The method of claim 7, wherein the Omi family polypeptide is selected from the group consisting of SEQ ID NOs. 44, 45, 48, 49, 54-57, 60-63, 66-75.

9. The method of claim 7, wherein the Omi family polypeptide is selected from the group consisting of Omi WT, OmiΔPDZ, OmiΔAVPS, Omi protease and Omi catalytic triad.

10. The method of claim 8, wherein the Omi family polypeptide is in a carrier.

11. The method of claim 10, wherein the carrier is a liposome.

12. A method for cleaving IAP comprising:
(a) isolating and amplifying an Omi family member gene;
(b) forming an Omi expression construct from the isolated and amplified Omi family member gene;
(c) transfecting a cell population having caspase bound by IAP, with the Omi family member construct; and,
(d) causing expression of the Omi vector, whereby the OMI family polypeptide cleaves IAP.

13. The method of claim 12, wherein the Omi family polypeptide is expressed by a nucleic acid sequence molecule selected from the group consisting of SEQ ID NOs. 1-3,6-8, 11-19, 22-27, and 30-39, and homologs thereof.

14. The method of claim 12, wherein expression is caused by the addition of etoposide.

15. The method of claim 12, wherein expression is caused by damage to the cell.

16. A method for cleaving IAP, wherein the method comprises:
contacting isolated IAP with an amount of an isolated Omi family polypeptide, whereby upon contact, the Omi family polypeptide will cleave the IAP.

17. A polypeptide for cleaving IAP selected from the group consisting of Omi WT, OmiΔPDZ, OmiΔAVPS, Omi protease, Omi catalytic triad, and homologs thereof.

18. The polypeptide of claim 17, comprising a carrier.

19. The polypeptide of claim 17, wherein the polypeptide is selected from the group consisting of expressed intra-cellular, isolated, or recombinant polypeptides.

20. An isolated polypeptide for cleaving IAP selected from the group consisting of SEQ ID NOs. 44, 45, 48, 49, 54-57, 60-63, 66-75, and homologs thereof.

21. A polypeptide for cleaving IAP comprising a protease domain selected from the group consisting of SEQ ID NOs. 64-66 and 75-80, and homologs thereof.

22. A polypeptide having increased protease activity comprising a polypeptide selected from the group consisting of SEQ ID NOs. 48, 49, 55, 56, 57, 60-63, 66-75, and homologs thereof.

23. A polypeptide for binding to a BIR site on an IAP, comprising SEQ ID NO. 77 and homologs thereof.

24. A polypeptide for cleaving IAP comprising a polypeptide selected from the group consisting of SEQ ID NOs. 44 and 45, and homologs thereof.

25. The polypeptide of claim 24, wherein the Omi to IAP molar ratio in vitro is equal to between 1:5 to 1:30 molar ratio of Omi to IAP.

26. A polypeptide which binds to IAP, but does not cleave IAP, selected from the group consisting of SEQ ID NOs. 46, 47, 50, 51, 58, 59, 64, 65, and homologs thereof.

27. A polypeptide which binds IAP but does not cleave IAP, comprising Omi SA.

28. An Omi serine protease for use in cleaving IAP.

29. An OmiΔPDZ for cleaving IAP.

30. A polypeptide molecule for cleaving an IAP comprising an amino acid sequence as set forth in C1ₙ₁-R1-C2ₙ₂-R2-C3ₙ₃-R3-C4ₙ₄, wherein:
(a) R1 is a serine;
(b) R2 is an amino acid residue selected from a group consisting of charged amino acid residues and aromatic amino acid residues;
(c) R3 is an amino acid residue selected from a group consisting of charged amino acid residues and polar amino acid residues; and,
(d) R1, R2 and R3 form a catalytic triad for cleavage of the IAP.

31. The molecule of claim 30, wherein R2 is an amino acid residue selected from a group consisting of histidine, lysine, arginine, phenylalanine, tyrosine, and tryptophan.

32. The molecule of claim 30, wherein R3 is an amino acid residue selected from a group consisting of aspartic acid, glutamic acid, lysine, histidine, and arginine.

33. The polypeptide of claim 30, wherein C1ₙ₁, C2ₙ₂, C3ₙ₃, and C4ₙ₄ are polypeptide chains.

34. The polypeptide of claim 33, wherein n1 is a number between 10 and 100.

35. The polypeptide of claim 33, wherein n2 is a number between 10 and 100.

36. The polypeptide of claim 33, wherein n3 is a number between 10 and 150.

37. The polypeptide of claim 33, wherein n4 is a number between 10 and 200.

38. The molecule of claim 33, wherein the C1ₙ₁ chain is the N-terminal and has an AVPS motif sequence that operably couples to IAP.

39. The molecule of claim 33, wherein the C4ₙ₄ chain is the C-terminal and has a hinge sequence and PDZ domain.

40. The molecule of claim 39, wherein the PDZ domain is removed.

41. The molecule of claim 30 wherein a C1ₙ₁ polypeptide chain has an N-terminal location and comprises an amino acid sequence as set forth in SEQ ID NOs. 54 and 55.

42. The polypeptide of claim 30 operably enclosed in a liposome in an aqueous medium.

43. A polypeptide molecule comprising an amino acid sequence as set forth in SEQ ID NOs. 54 and 55.

44. A serine protease polypeptide molecule wherein the molecule is of the formula comprising C1ₙ₁-R1-C2ₙ₂-R2-C3ₙ₃-R3-C4ₙ₄, wherein:
(a) R1 is a serine;
(b) R2 is an amino acid residue selected from a group consisting of a charged amino acid residue and an aromatic amino acid residue;
(c) R3 is an amino acid residue selected from a group consisting of a charged amino acid residue and a polar amino acid residue;
(d) C1ₙ₁, C2ₙ₂, C3ₙ₃, and C4ₙ₄ are polypeptide chains;
(e) n1 is an amino acid residue number ranging between 10 and 100;
(f) n2 is an amino acid residue number ranging between 10 and 100;
(g) n3 is an amino acid residue number ranging between 10 and 150;
(h) n4 is an amino acid residue number ranging between 10 and 200; and,
(i) R1, R2 and R3 form a catalytic triad for cleavage of a polypeptide substrate.

45. A polypeptide molecule comprising an amino acid sequence selected from the group consisting of SEQ ID NOs. 46, 47, and homologs thereof.

46. A polypeptide molecule comprising an amino acid sequence selected from the group consisting of SEQ ID NOs. 48, 49 and homologs thereof.

47. A polypeptide molecule comprising an amino acid sequence selected from the group consisting of SEQ ID NOs. 56, 57 and homologs thereof.

48. A polypeptide molecule comprising an amino acid sequence selected from the group consisting of SEQ ID NOs. 60 through 63 and homologs thereof.

49. A polypeptide molecule for inhibiting IAP cleavage comprising an amino acid sequence as set forth in C1ₙ₁-R1-C2ₙ₂-R2-C3ₙ₃-R3-C4ₙ₄, wherein:
(a) R₁ is an amino acid residue selected from a group consisting of an alanine, an arginine, an aspartic acid, an asparagine, a cysteine, a glutamic acid, a glutamine, a glycine, a histidine, an isoleucine, a leucine, a lysine, a methionine, a phenylalanine, a proline, a threonine, a tryptophan, a tyrosine, and a valine;
(b) R₂ is a histidine;
(c) R₃ is an aspartic acid; and,
(d) an AVPS moiety binds to IAP.

50. An OmiΔPDZ expressed intra-cellularly by an OmiΔPDZ vector in a cell.

51. A nucleic acid sequence, which expresses a polypeptide which cleaves IAP, wherein the nucleic acid sequence is selected from the group consisting of Omi family member nucleic acid sequences, degenerate variants of the Omi family member, and homologous sequences to the Omi family member.

52. The nucleic acid sequence of claim 51, wherein the sequence is selected from the group consisting of SEQ ID NOs. 1-41, and homologos sequences thereof.

53. The nucleic acid sequence of claim 51, wherein the sequence is selected from the group consisting of Omi WT, OmiΔPDZ, OmiΔAVPS, Omi protease, Omi catalytic triad, and homologs thereof.

54. A nucleic acid sequence which expresses isolated polypeptide for cleaving IAP, wherein the nucleic acid sequence is selected from the group consisting of SEQ ID NOs. 1-43, and homologos sequences thereof.

55. A nucleic acid sequence which expresses a polypeptide for cleaving IAP, wherein the nucleic acid sequence expresses a protease domain, selected from the group consisting of SEQ ID NOs. 44, 45, 48 and 49, and homologos sequences thereof.

56. A nucleic acid sequence which expresses a polypeptide having increased protease activity comprising a polypeptide selected from the group consisting of SEQ ID NOs. 48, 49, 50, 56, 57, 58, 59, 60, 61, and homologos sequences thereof.

57. A nucleic acid sequence which expresses a polypeptide for binding to a BIR site on an IAP, comprising SEQ ID NO. 82 and homologos sequences thereof.

58. A nucleic acid sequence which expresses a polypeptide for cleaving IAP comprising a polypeptide selected from the group consisting of SEQ ID NOs. 44, 45, and homologous sequences thereof.

59. A nucleic acid sequence, which expresses a polypeptide, which binds to IAP, but does not cleave IAP, selected from the group consisting of SEQ ID NOs. 4, 5, 9, 10, 20, 21, 28, and 29, and homologs thereof.

60. A nucleic acid sequence which expresses a polypeptide which binds IAP but does not cleave IAP, comprising Omi SA.

61. An expression vector comprising a nucleic acid that expresses a molecule for cleaving IAP selected from a group consisting of SEQ ID NOs. 1-43, and homologous sequences thereof.

62. The expression vector of claim 61, wherein the expression vector is selected from a group consisting of a plasmid and an episome.

63. The expression vector of claim 61, wherein the expression vector comprises a replicating virus.

64. The expression vector of claim 61, wherein the expression vector is a pE721b vector.

65. An Omi expression vector, comprising an Omi family nucleic acid sequence and a vector selected from the group consisting of eukaryotic vectors MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pET21b, pCEP4, and pEBVHis vectors.

66. A transfected cell comprising:
(a) an expression vector that expresses an Omi family member polypeptide; and,
(b) a promoter.

67. The transfected cell of claim 66, wherein the transfected cell is selected from the group consisting of an animal cell and a plant cell.

68. The transfected cell of claim 66, wherein the transfected cell is a tumor cell.

69. The transfected cell of claim 66, wherein the polypeptide is an OmiΔPDZ.

70. The transfected cell of claim 66, wherein the vector is selected from the group consisting of eukaryotic vectors MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pET21b, pCEP4, and pEBVHis vectors.

71. A pharmaceutical composition for treatment of a hyperproliferative disorder in an animal which comprises a pharmacologically acceptable carrier and a therapeutically effective amount of the liposomes containing Omi family member polypeptides of SEQ ID NOs. 44, 45, 48, 49, 52-57, 60-63, and 66-75.

72. A method of treating the hyperproliferative disorder comprising administering an effective amount of the pharmacological composition of claim 71 into an animal.

73. A hybridization kit for detecting an Omi wild-type gene, wherein the kit comprises:
(a) a container; and,
(b) a nucleic acid molecule comprising a nucleotide molecule selected from a group consisting of Omi family nucleic acid sequences.

74. A hybridization kit for detecting an Omi mutant gene, wherein the kit comprises:
(a) a container; and,
(b) a nucleic acid molecule comprising a molecule selected from a group consisting of SEQ ID NOs. 1-41, and homologous sequences thereof.

75. A kit for detecting an Omi gene comprising:
(a) PCR primers spanning an Omi family gene, a positive control; and,
(b) sequencing products.

76. A kit for detecting an Omi polypeptide, wherein the kit comprises:
(a) a container; and,
(b) an antibody derived from polypeptide selected from a group consisting of SEQ ID NOs. 44-77, and homologs thereof.

77. An antibody which binds to the Omi serine.

78. An antibody which binds to a protease.

79. A mammalian cell consisting essentially of:
(a) a cell transfected by an Omi expression vector;
(b) the transfected cell producing an IAP-cleaving molecule selected from the group consisting of said IAP-cleaving molecules; and,
(c) a promoter controlling transcription and the quantity of production of said IAP-cleaving molecule.

80. The mammalian cell of claim 79, wherein, in the transfected cell, the expression vector is autonomously replicating.

81. The mammalian cell of claim 79, wherein said transfected cell is a human cell.

82. A method of detecting and identifying an IAP-cleaving molecule comprising:
(a) binding an antibody directed against an antigen associated with the IAP-cleavage site on said IAP-cleaving molecule to a solid phase adsorbent surface;
(b) adding a specimen containing a plurality of unlabelled IAP-cleaving molecules;
(c) adding a plurality of labeled IAP-cleaving molecules;
(d) detecting the bound labeled IAP-cleaving molecules; and,
(e) calculating the amount of bound unlabeled IAP-cleaving molecules.

83. The method of claim 82, wherein the antibody is selected from the group consisting of a monoclonal antibody, a polyclonal antibody, and a recombinant antibody.

84. The method of claim 82, wherein the label is selected from the group consisting of a radiolabel, a luminescent label, and a colorimetric label.

85. The method of claim 82, wherein the antibody is selected of the group consisting of SEQ ID NOs. 44-77, and homologs thereof.

86. An Omi expression vector, comprising an Omi family nucleic acid sequence selected from the group consisting of SEQ ID NOs. 1-41, and homologs thereof, and a vector selected from the group consisting of eukaryotic vectors MSCV, Harvey murine sarcoma virus, pFastBac, pFastBac HT, pFastBac DUAL, pSFV, pTet-Splice, pEUK-C1, pPUR, pMAM, pMAMneo, pBI101, pBI121, pDR2, pCMVEBNA, YACneo, pSVK3, pSVL, pMSG, pCH110, pKK232-8, p3'SS, pBlueBacIII, pCDM8, pcDNA1, pZeoSV, pcDNA3, pREP4, pET21b, pCEP4, and pEBVHis vectors.
